(19) United States
(12) Patent Application Publication
(10) Pub. No.: US 2022/0380727 A1
(43) Pub. Date: Dec. 1, 2022

(54) GENERATION OF ENGINEERED REGULATORY T CELLS

(71) Applicant: Sangamo Therapeutics, Inc., Brisbane, CA (US)

(72) Inventors: Anthony Conway, Emeryville, CA (US); Helen Fong, Oakland, CA (US); George Kwong, San Jose, CA (US)

(73) Assignee: Sangamo Therapeutics, Inc., Brisbane, CA (US)

(21) Appl. No.: 17/775,016

(22) PCT Filed: Nov. 9, 2020
(86) PCT No.: PCT/US2020/059730

§ 371 Date: May 6, 2022

Related U.S. Application Data
(60) Provisional application No. 62/933,252, filed on Nov. 8, 2019.

(51) Int. Cl. C12N 5/0783; C12N 15/90; C12N 15/86; A61K 35/17; C07K 14/47

(52) U.S. Cl. CPC C12N 5/0637; C12N 15/907; C12N 15/86; A61K 35/17; C07K 14/4705; C12N 2506/45; C12N 2750/14143; C12N 2502/1394; C12N 2501/2302

(57) ABSTRACT

Provided herein are genetically engineered mammalian stem and progenitor cells that have increased potential to differentiate into regulatory T cells. Also provided are methods of making and use thereof.

### CROSS REFERENCE TO RELATED APPLICATION

The present application claims priority from U.S. Provisional Application 62/933,252, filed on Nov. 8, 2019, the content of which is incorporated herein by reference in its entirety.

### BACKGROUND

A healthy immune system is one that is in balance. Cells involved in adaptive immunity include B and T lymphocytes. There are two general types of T lymphocytes-effector T (Teff) cells and regulatory T (Treg) cells. Teff cells include CD4+T helper cells and CD8+ cytotoxic T cells. Teff cells play a central role in cellular-mediated immunity following antigen challenge. A key regulator of the Teff cells and other immune cells is the Treg cells, which prevent excessive immune responses and autoimmunity (see, e.g., Romano et al., Front Immunol. (2019) 10, art. 43).

Some Tregs are generated in the thymus; they are known as natural Treg (nTreg) or thymic Treg (tTreg). Other Tregs are generated in the periphery following an antigen encounter or in cell culture, and are known as induced Tregs (iTreg) or adaptive Tregs. Tregs actively control the proliferation and activation of other immune cells, including inducing tolerance, through cell-to-cell contact involving specific cell surface receptors and the secretion of inhibitory cytokines such as IL-10, TGF-0 and IL-35 (Dominguez-Villar and Hafler, Nat Immunol. (2018) 19:665-73). Failure to induce tolerance can lead to autoimmunity and chronic inflammation. Loss of tolerance can be caused by defects in Treg functions or insufficient Treg numbers, or by unresponsive or over-activated Teff (Sadlon et al., Clin Transl Immunol. (2018) 7:e1011, doi:10-1002/cti2.1011).

In recent years, there has been much interest in the use of Tregs to treat diseases. A number of approaches, including adoptive cell therapy, have been explored to boost Treg numbers and functions in order to treat autoimmune diseases. Treg transfer, which delivers an activated and expanded population of Tregs, has been tested in patients with autoimmune diseases such as type I diabetes, cutaneous lupus erythematosus, and Crohn's disease, and in organ transplantation (Dominguez-Villar, supra; Safinia et al., Front Immunol. (2018) 9:354).

Currently, the only sources of Tregs for cell therapies are adult or adolescent primary blood (e.g., whole blood or apheresis products) and tissue (e.g., thymus). Isolation of Tregs from these sources is invasive and time-consuming, and yields only small numbers of Tregs. Further, Tregs obtained from these samples are polyclonal in nature and can introduce variability in their potential immunosuppressive response. There also is evidence that simply increasing the number of Tregs may not be sufficient to control disease (McGovern et al., Front Immunol. (2017) 8, art. 1517). Engineered monoclonal Tregs with antigen-specific moieties, such as CARs or engineered TCRs, may allow for enhanced immunomodulatory response at the site of autoimmune activity or organ transplant. There remains a need for efficiently obtaining genetically engineered, monoclonal Treg cells in large numbers.

### SUMMARY

The present disclosure provides methods and compositions for promoting differentiation of stem cells, including induced pluripotent stem cells (iPSCs) and progenitor cells, into regulatory T cells. In preferred embodiments, the engineered regulatory T cells are prepared for adoptive cell therapy.

In one aspect, the present disclosure provides a genetically engineered mammalian cell (e.g., a human cell) comprising a heterologous sequence in the genome, wherein the heterologous sequence comprises a transgene encoding a lineage commitment factor (also termed lineage induction factor herein), and wherein the lineage commitment factor promotes the differentiation of the cell to a CD4⁺ regulatory T cell (Treg) or promotes the maintenance of the cell as a CD4⁺ Treg. In some embodiments, the heterologous sequence is integrated into a safe harbor site in the genome of the engineered cell (e.g., the AAVS1 gene locus). In other embodiments, the heterologous sequence is integrated into a T cell specific gene locus, i.e., a locus containing a gene that is specifically expressed in T cells, such as Tregs (e.g., the FOXP3 site and the Helios site); in these embodiments, the transgene may be under the control of transcription-regulatory elements in the gene locus.

In another aspect, the present disclosure provides a method of making a genetically engineered mammalian cell, comprising: contacting a mammalian cell with a nucleic acid construct comprising (i) a heterologous sequence and (ii) a first homologous region (HR) and a second HR flanking the heterologous sequence, wherein the heterologous sequence comprises a transgene, the first and second HRs are homologous to a first genomic region (GR) and a second GR, respectively, in a T cell specific gene locus or a genomic safe harbor in the mammalian cell; and culturing the cell under conditions that allow integration of the heterologous sequence between the first and second GRs in the T cell specific gene locus or genomic safe harbor. In some embodiments, the heterologous sequence integration is facilitated by a zinc finger nuclease or nickase (ZFN), a transcription activator-like effector domain nuclease or nickase (TALEN), a meganuclease, an integrase, a recombinase, a transposase, or a CRISPR/Cas system. In some embodiments, the nucleic acid construct is a lentiviral construct, an adenoviral construct, an adeno-associated viral construct, a plasmid, a DNA construct, or an RNA construct.

In some embodiments, the transgene comprises a coding sequence for an additional polypeptide, wherein the coding sequence for the lineage commitment factor and the coding sequence for the additional polypeptide are separated by an in-frame coding sequence for a self-cleaving peptide or by an internal ribosome entry site (IRES). In particular embodiments, the additional polypeptide is another lineage commitment factor, a therapeutic protein, or a chimeric antigen receptor (CAR).

In some embodiments, the heterologous sequence is integrated into an exon in the T cell specific gene locus and comprises: an internal ribosome entry site (IRES) immediately upstream of the transgene; or a second coding sequence for a self-cleaving peptide immediately upstream of and in-frame with the transgene. In further embodiments, the heterologous sequence further comprises, immediately upstream of the IRES or the second coding sequence for a self-cleaving peptide, a nucleotide sequence comprising all the exonic sequences of the T cell specific gene locus that are downstream of the integration site, such that the T cell specific gene locus remains able to express an intact T cell specific gene product. In particular embodiments, the T cell specific gene locus is a T cell receptor alpha constant (TRAC) gene locus, and the heterologous sequence is optionally integrated into exon 1, 2, or 3 of the TRAC gene locus.

In some embodiments, the transgene encodes FOXP3, Helios, or ThPOK. In further embodiments, the transgene comprises a coding sequence for FOXP3 and a coding sequence of ThPOK, wherein these two coding sequences are in-frame and are separated by an in-frame coding sequence for a self-cleaving peptide.

In some embodiments, the cell is a human cell. In further embodiments, the cell is a stem or progenitor cell, optionally selected from embryonic stem cell, induced pluripotent stem cell, mesodermal stem cell, mesenchymal stem cell, hematopoietic stem cell, a lymphoid progenitor cell, or a progenitor T cell. In some embodiments, the cell is reprogrammed from a T cell (e.g., a Treg, a CD4⁺ T cell, or a CD8⁺ T cell). In some embodiments, the engineered cell is a Treg.

In some embodiments, the present disclosure provides a method of producing the engineered Treg, the method comprising: culturing the engineered stem or progenitor cell herein in a tissue culture medium that comprises (i) a low IL-2 dose, (ii) an inhibitor of IL-7Ra (CD27) signaling (e.g., an antibody), (iii) an inhibitor of CCR7 signaling (e.g., an antibody). In some embodiments, the present disclosure provides a method of producing the engineered Treg, the method comprising: co-culturing the engineered stem or progenitor cell herein with MS5-DLL1/4 stromal cells; OP9 or OP9-DLL1 stromal cell; or EpCAM⁻CD56⁺ stromal cells. The present disclosure provides also Treg cells obtained by these methods.

In some embodiments, the engineered cells further comprise a null mutation in a gene selected from a Class II major histocompatibility complex transactivator (CIITA) gene, an HLA Class I or II gene, a transporter associated with antigen processing, a minor histocompatibility antigen gene, and a β2 microglobulin (B2M) gene.

In some embodiments, the engineered cells further comprise a suicide gene optionally selected from a HSV-TK gene, a cytosine deaminase gene, a nitroreductase gene, a cytochrome P450 gene, or a caspase-9 gene.

The present disclosure further provides a method of treating a patient (e.g., a human patient) in need of immunosuppression, comprising administering to the patient the engineered cell (e.g., engineered Tregs) provided herein. Also provided are use of the engineered cells herein in the manufacture of a medicament in treating a patient (e.g., a human patient) in need of immunosuppression, as well as the engineered cells herein for use in treating a patient (e.g., a human patient) in need of immunosuppression. In some embodiments, the patient has an autoimmune disease or has received or will receive tissue transplantation.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic diagram depicting a genome editing approach to integrating a transgene encoding one or more Treg commitment (or induction) factors (“TFs”) into exon 2 of the human TRAC gene. A zinc finger nuclease (ZFN) produced from an introduced mRNA makes a double-stranded break at a specific site (lightning bolt) in exon 2. The donor sequence, introduced by an adeno-associated virus (AAV) 6 vector, contains, from 5′ to 3′: homology region 1; a coding sequence for self-cleaving peptide T2A; a coding sequence for a fusion of a first TF, self-cleaving peptide P2A, a second TF2, self-cleaving peptide E2A and a third TF3; a poly-adenylation (polyA) signal sequence; and homology region 2. The homology regions are homologous to the genomic regions flanking the ZFN cleavage site. The TRAC exon 2 portion upstream of the integration site, the T2A coding sequence, and the TF coding sequence(s) are in-frame with each other. Under this approach, expression of the TRAC protein is knocked out as a result of the transgene integration. Expression of the integrated sequences is regulated by the endogenous TCR alpha chain promoter.

FIG. 2 is a schematic diagram depicting a genome editing approach similar to the one depicted in FIG. 1, but here, the heterologous sequence comprises a partial TRAC cDNA encompassing the TRAC exonic sequences downstream of the integration site (i.e., the exon 2 sequence 3′ to the integration site and the exon 3 sequence). This partial TRAC cDNA is placed immediately upstream of, and in-frame, with the T2A coding sequence, such that the engineered locus expresses an intact TCR alpha chain and TF(s) under the endogenous TCR alpha chain promoter.

FIG. 3 is a schematic diagram depicting yet another genome editing approach to integrating a transgene encoding one or more commitment factors. In this approach, the transgene is integrated into a genomic safe harbor. In this figure, the transgene is inserted into intron 1 of the human AAVS1 gene locus and linked operably to a doxycycline (Dox) inducible promoter. SA: splice acceptor. 2A: coding sequence for self-cleaving peptide 2A. PuroR: puromycin-resistant gene. TI: targeted integration.

FIG. 4 is a panel of graphs showing data generated from cells edited using the schematic outlined in FIG. 3. The transgene encodes a green fluorescent protein (GFP). Puro: puromycin. Dox: doxycycline.

FIG. 5 is a schematic diagram depicting a genome editing approach in which a transgene encoding one or more commitment factors is integrated into intron 1 of the human AAVS1 gene. The heterologous sequence integrated into the genome includes a CAR-encoding sequence. Once Treg differentiation is accomplished, the transgene encoding the commitment factor (placed between the two LoxP sites) is excised, leaving only the CAR expression cassette at the integration site.

FIG. 6 is a schematic diagram depicting a genome editing approach to integrating a transgene encoding one or more commitment factors into exon 2 of the human TRAC gene. In this approach, the heterologous sequence integrated into the genome includes a CAR-encoding sequence. Once Treg differentiation is accomplished, the transgene encoding the commitment factor (placed between the two LoxP sites) is excised, leaving only the CAR expression cassette at the integration site.

FIG. 7 is a schematic diagram depicting a process for reprogramming mature Tregs having a single rearranged TCR to inducible pluripotent stem cells (iPSCs). Following expansion, the iPSCs are re-differentiated back into a Treg phenotype. The TCR here targets an antigen that is not an allo-antigen.

FIG. 8 is a schematic diagram depicting a process in which an iPSC differentiates into a Treg. HSC: hematopoietic stem cell. Single positive: CD4⁺ or CD8⁺. Double positive: CD4⁺CD8⁺.

FIG. 9 is a panel of cell sorting graphs demonstrating that introduction of an antibody for the alpha unit of the IL-7 receptor (IL-7Ra) to tissue culture media skews the differentiation of iPSC-derived progenitor T cells from forming CD8 single positive cells (top left quadrants) to forming CD4 single positive cells (bottom right quadrants). The antibody was added to tissue culture media at three concentrations (low, medium, and high). This effect was shown in two separate experiments (Expt. #1 and Expt. #2).

FIG. 10 is a schematic diagram depicting multiple processes for differentiating iPSCs into Tregs. The cells are cultured on Lymphocyte Differentiation Coating Material (feeder independent) or with OP9 stromal cells or OP9-DLL1 stromal cells (OP9 cells expressing the Notch ligand, Delta-like 1) stromal cells (feeder dependent). The cells are then further cultured as depicted in FIG. 8 to promote differentiation into Tregs. In an alternative path, the three-dimensional embryonic mesodermal organoids (EMO) are formed by co-culturing iPSCs with MSS-DLL1/4 or EpCAM⁻CD56⁺ stromal cells; after hematopoietic induction of the EMO, artificial thymic organoids (ATO) are formed, which are induced to generate mature Tregs with a TCR repertoire more akin to thymically selected Tregs.

FIG. 11 is a schematic diagram depicting a genome editing approach to integrating either a CRISPR activation (CRISPRa) or inihibition (CRISPRi) library, which includes either a dead Cas9 (dCas9) fused to either the VPH activating domain or KRAB inhibition domain, respectively. In this figure, the library (transgene) is integrated into intron 1 of the human AAVS1 gene.

FIG. 12 is a panel of graphs comparing the ability to generate T cells between iPSCs derived from naïve regulatory, CD4⁺, and CD8⁺ T cells (collectively TiPSCs) and iPSCs derived from CD34⁺ cells. Panel A shows the percentage of live/single cells that co-expressed CD3 and TCRαβ during differentiation in the TiPSCs and the CD34-derived iPSCs. Panel B is panel of representative flow cytometry plots depicting the expression of CD3 and TCRαβ in differentiating T cells from the iPSCs. The types and distribution of cells from CD3⁺ TCRαβ⁺ cells (Panel C) as well as from live/single cells (Panel D) from each iPSC line were also examined. CD4sp: CD4 single positive. CD8sp: CD8 single positive. DN: double negative (CD4⁻CD8⁻). DP: double positive (CD4⁺CD8⁺). Statistical significance was determined by unpaired t-test with Welch's correction. Asterisks indicate statistical significance.

FIG. 13A is a panel of flow cytometry plots showing the expression of FOXP3 and anti-HLA-A2 chimeric antigen receptor (CAR) in T cells derived from iPSC lines edited at exon 2 of the TRAC locus in an approach illustrated in FIG. 2. The transgenes were FOXP3/Helios/CAR, FOXP3/CAR, FOXP3, or GFP.

FIG. 13B is a graph showing cytokine secretion analysis of the cells in the study of FIG. 13A.

### DETAILED DESCRIPTION

Pluripotent stem cells (PSCs) can be expanded indefinitely and give rise to any cell type within the human body. PSCs (e.g. human embryonic stem cells and induced pluripotent stem cells) represent an ideal starting source for producing large numbers of differentiated cells for therapeutic applications. The present disclosure provides methods of generating Treg cells from PSCs such as induced PSCs (iPSCs). Also included in the present disclosure are methods of generating Treg cells from multipotent cells such as mesodermal progenitor cells, hematopoietic stem cells, or lymphoid progenitor cells. Multipotent cells, including multipotent stem cells and tissue progenitor cells, are more limited in their ability to differentiate into different cell types as compared to pluripotent cells.

In the present methods, stem cells and/or progenitor cells are genetically engineered to overexpress (i.e., express at a level higher than the cell normally would) Treg lineage commitment factors (e.g., FOXP3, Helios, Ikaros) and/or CD4⁺ helper T cell lineage commitment factors (e.g., Gata3 and ThPOK). These factors facilitate the differentiation of the engineered stem and/or progenitor cells into Tregs. These factors may be constitutively overexpressed during the entire or part of the Treg differentiation process; or may be inducibly expressed during a specific period of the Treg differentiation process (e.g., via doxycycline-induced TetR-mediated gene expression).

In some embodiments, the commitment factors are encoded by transgene(s) randomly integrated into the genome of the stem or progenitor cells (e.g., by using a lentiviral vector, a retroviral vector, or a transposon).

Alternatively, the commitment factors are encoded by transgene(s) that are integrated into the genome of the stem or progenitor cells in a site-specific manner. For example, the transgenes are integrated at a genomic safe harbor site, or at a genomic locus of a T cell specific gene, such as the T cell receptor alpha chain constant region (i.e., T cell receptor alpha constant or TRAC) gene. In the former approach, the transgene can be optionally placed under the transcription control of a T cell specific promoter or an inducible promoter. In the latter approach, the transgene can be expressed under the control of the endogenous promoter and other transcription-regulatory elements for the T cell specific gene (e.g., the TCR alpha chain promoter). An advantage of placing the transgene under the control of a T cell specific promoter is that the transgene will only be expressed in T cells, as it is intended to be, thereby improving the clinical safety of the engineered cells.

In some embodiments, the present methods may additionally include tissue culture steps that further promote this differentiation.

Regulatory T cells maintain immune homeostasis and confer immune tolerance. The engineered Treg cells, which may be autologous or allogeneic, can be used in cell-based therapy to treat patients in need of induction of immune tolerance or restoration of immune homeostasis, such as patients receiving organ transplantation or allogeneic cell therapy and patients with an autoimmune disease. The present Treg cells will have improved therapeutic efficacy because they can be monoclonal, avoiding the variability caused by polyclonality in past Treg therapies. Further, the Treg cells may be selected based on their antigen specificity. For example, Treg cells may be selected for expressing a T cell receptor (TCR) or an edited-in chimeric antigen receptor (CAR) specific for an antigen at an in vivo site where Tregs are desired such that the TCR or CAR directs the Treg cells to the site (e.g., site of inflammation), thereby enhancing the potency of the cells.

### I. Transgenes Encoding CD4⁺ Treg Commitment Factors

To promote the differentiation of progenitor cells or stem cells such as iPSCs into Tregs, the cells may be engineered to express one or more proteins that promote the lineage commitment of the progenitor or stem cells to become CD4⁺ helper T cells and ultimately Treg cells. As used herein, the terms “regulatory T cells,” “regulatory T lymphocytes,” and “Tregs” refer to a subpopulation of T cells that modulates the immune system, maintains tolerance to self-antigens, and generally suppresses or downregulates induction and proliferation of T effector cells. The Treg phenotype is in part dependent on the expression of the master transcription factor forkhead box P3 (FOXP3), which regulates the expression of a network of genes essential for immune suppressive functions (see, e.g., Fontenot et al., Nature Immunology (2003)4(4):330-6). Tregs often are marked by the phenotype of CD4⁺CD25⁺CD127^{lo}FOXP3⁺. In some embodiments, Tregs are also CD45RA⁺, CD62L^{hi} Helios*, and/or GITR⁺. In particular embodiments, Tregs are marked by CD4⁺CD25⁺CD127^{lo}CD62L⁺ or CD4⁺CD45RA⁺CD25^{hi}CD127^{lo}.

In the present methods, transgenes that are introduced to the genome of the stem cells or progenitor cells to promote their differentiation to Tregs may be, without limitations, those encoding one or more of CD4, CD25, FOXP3, CD4RA, CD62L, Helios, GITR, Ikaros, CTLA4, Gata3, Tox, ETS1, LEF1, RORA, TNFR2, and ThPOK. The cDNA sequences encoding these proteins are available at GenBank and other well-known gene databases. Expression of one or more of these proteins will help commit the stem or progenitor cells to the Treg fate during differentiation. In some embodiments, the transgene encodes the Treg lineage commitment factor FOXP3 and/or the CD4⁺ helper T cell lineage commitment factor ThPOK (He et al., Nature (2005) 433(7028):826-33). In some embodiments, the transgene encodes Helios, which is expressed in a subpopulation of Tregs (Thornton et al., Eur J Immunol. (2019) 49(3):398-412).

In some embodiments, the stem or progenitor cells may be engineered to overexpress commitment factors that enhance hematopoietic stem cell (HSC) multipotency (see Sugimura et al., Nature (2017) 545(7655):432-38). These factors include, without limitation, HOXA9, ERG, RORA, SOX4, LCOR, HOXA5, RUNX1, and MYB.

In some embodiments, the stem or progenitor cells may be engineered to downregulate EZHI via an engineered site-specific transcriptional repression construct (e.g. ZFP-KRAB, CRISPRi, etc.), shRNA, or siRNA to enhance HSC multipotency (see Vo et al., Nature (2018) 553(7689):506-510).

### II. Integration of Transgenes Encoding Commitment Factors

To engineer stem cells or progenitor cells genetically, a heterologous nucleotide sequence carrying a transgene of interest is introduced into the cells. The term “heterologous” here means that the sequence is inserted into a site of the genome where this sequence does not naturally occur. In some embodiments, the heterologous sequence is introduced into a genomic site that is specifically active in Treg cells. Examples of such sites are the genes encoding a T cell receptor chain (e.g., TCR alpha chain, beta chain, gamma chain, or delta chain), a CD3 chain (e.g., CD3 zeta, epsilon, delta, or gamma chain), FOXP3, Helios, CTLA4, Ikaros, TNFR2, or CD4.

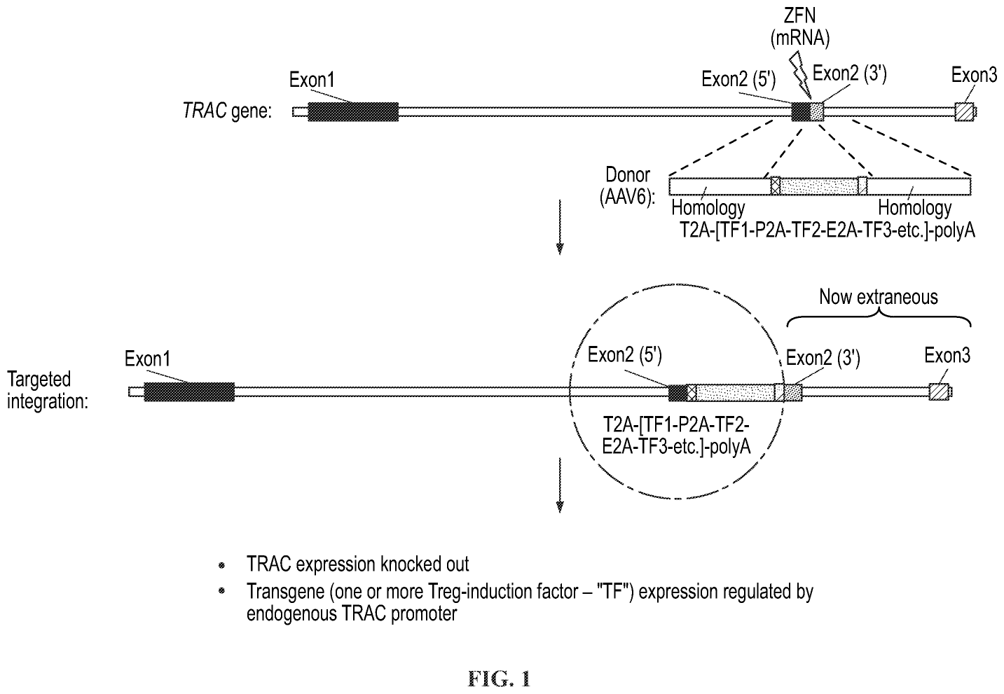

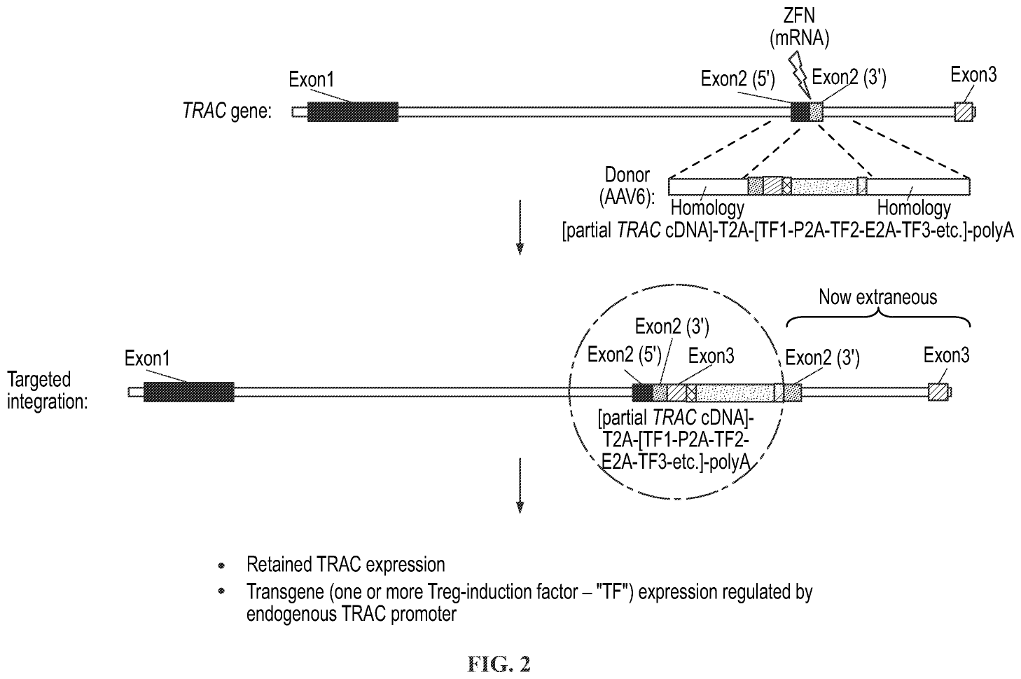

By way of example, the heterologous sequence is introduced into one or both TRAC alleles in the genome. The genomic structure of the TRAC locus is illustrated in FIGS. 1 and 2. The TRAC gene is downstream of the TCR alpha chain V and J genes. TRAC contains three exons, which are transcribed into the constant region of the TCR alpha chain. The gene sequence and the exon/intron boundaries of the human TRAC gene can be found in Genbank ID 28755 or 6955. The targeted site for integration may be, for example, in an intron (e.g., intron 1 or 2), in a region downstream of the last exon of the TRAC gene, in an exon (e.g., exon 1, 2, or 3), or at a junction between an intron and its adjacent exon.

FIGS. 1 and 2 illustrate two different approaches to targeting a heterologous sequence into exon 2 of the human TRAC locus through gene editing. In both approaches, the transgene encodes a polypeptide containing one or more Treg commitment or induction factors (e.g., FOXP3), separated by a self-cleaving peptide (e.g., P2A, E2A, F2A, T2A). In some embodiments, the FOXP3 transgene is engineered to convert lysine residues, which are known to become acetylated, into arginine residues (e.g., K31R, K263R, K268R), so as to enhance Treg suppressive activity (see Kwon et al., J Immunol. (2012) 188(6):2712-21).

In the approach depicted in FIG. 1, the expression of TCR alpha chain in the engineered cell is disrupted by the insertion of the heterologous sequence. In this approach, the heterologous sequence integrated into the genome contains, from 5′ to 3′, (i) a coding sequence for self-cleaving peptide T2A (or an internal ribosome entry site (IRES) sequence), (ii) a coding sequence for the commitment factor(s), and (iii) a polyadenylation (polyA) site. Once integrated, the engineered TRAC locus will express the commitment factor(s) under the endogenous promoter, where the T2A peptide allows the removal of any TCR alpha chain sequence from the first commitment factor (i.e., any TCR variable domain sequence, as well as any constant region sequence encoded by exon 1 and the portion of exon 2 5′ to the integration site). Because the TRAC gene is disrupted, no functional TCR alpha chain can be produced in the engineered cell. Due to the inclusion of a P2A coding sequence in the transgene, the engineered locus can express all individual Treg induction factor(s) as separate polypeptides. Under this approach, the stem or progenitor cells may be further engineered to express a desired antigen-recognition receptor (e.g., a TCR or CAR targeting an antigen of interest).

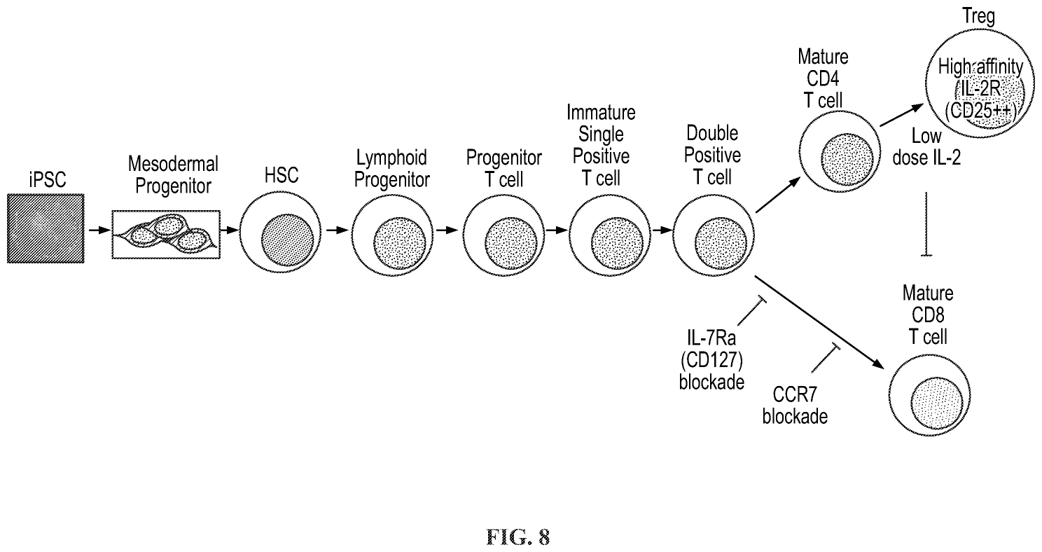

In the approach depicted in FIG. 2, the heterologous sequence may contain, from 5′ to 3′, (i) the TRAC exonic sequences 3′ to the integration site (i.e., the remaining exon 2 sequence downstream of the integration site, and the entire exon 3 sequence), (ii) a coding sequence for T2A (or IRES sequence), (iii) a coding sequence for one or more commitment factors, and (iv) a polyA site. The inclusion of the TRAC exonic sequences and T2A in the heterologous sequence will allow the production of an intact TCR alpha chain. The inclusion of P2A will allow the production of the commitment factor(s) as separate polypeptides. The TCR alpha chain, the exogenously introduced commitment factor(s) are all expressed under the control of the endogenous TCR alpha chain promoter. This approach is particularly suitable for engineering iPSCs reprogrammed from a mature Treg that has already rearranged its TCR alpha and beta chain loci (see FIG. 7 and discussions below). Tregs differentiated from such genetically engineered iPSCs will retain the antigen specificity of the ancestral Treg cell. Further, retention of the TCR alpha chain expression may yield enhanced T cell and Treg differentiation since TCR signaling is integrally involved in T cell and Treg development in the thymus.

In alternative embodiments, the transgene may be integrated into a TRAC intron, rather than a TRAC exon. For example, the transgene is integrated in an intron upstream of exon 2 or exon 3. In such embodiments, the heterologous sequence carrying the transgene may contain, from 5′ to 3′, a splice acceptor (SA) sequence, the transgene encoding one or more Treg commitment factors, and a polyA site. Where the expression of a rearranged TCR alpha chain gene is desired, the heterologous sequence may contain, from 5′ to 3′, (i) an SA sequence, (ii) any exon(s) downstream of the heterologous sequence integration site, (iii) a coding sequence for a self-cleaving peptide or an IRES sequence, (iv) the transgene encoding one or more commitment factors, and (v) a polyA site. Once integrated, the SA will allow the expression of an RNA transcript encoding an intact (i.e., full-length) TCR alpha chain, the self-cleaving peptide, and the commitment factor(s). Translation of this RNA transcript will yield two (or more) separate polypeptide products—the intact TCR alpha chain and the one or more commitment factors. Examples of SA sequences are those of the TRAC exons and other SA sequences known in the art.

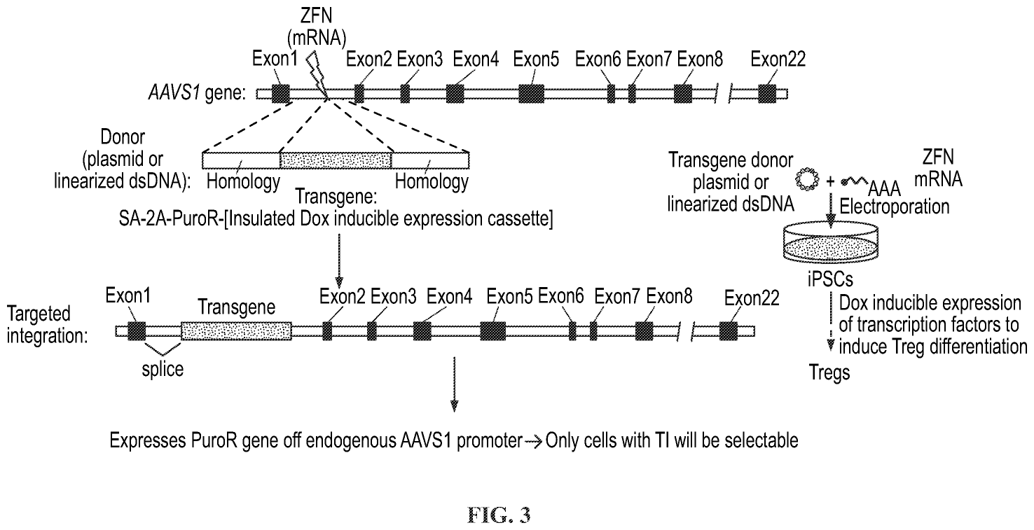

In some embodiments, the transgene is integrated into a genomic safe harbor of the engineered cells. Genomic safe harbor sites include, without limitation, the AAVS1 locus; the ROSA26 locus; the CLYBL locus; the gene loci for albumin, CCR5, and CXCR4; and the locus where the endogenous gene is knocked out in the engineered cells (e.g., the T cell receptor alpha or beta chain gene locus, the HLA gene locus, the CIITA locus, or the β2-microglobulin gene locus). FIG. 3 illustrates such an approach. In this example, the heterologous sequence is integrated into the human AAVS1 gene locus at, e.g., intron 1. Expression of the commitment factor encoding transgene is controlled by a doxycycline-inducible promoter. The doxycycline-inducible promoter may include a 5-mer repeat of the Tet-responsive element. Upon the introduction of doxycycline to tissue culture, the constitutively expressed inducible form of the tetracycline-controlled transactivator (rtTA) binds to the Tet-responsive element and initiate transcription of the commitment factor(s). A zinc finger nuclease (ZFN) produced from an introduced mRNA makes a double-stranded break at a specific site (lightning bolt) in intron 1. The donor sequence, introduced by plasmid DNA or linearized double-stranded DNA, contains, from 5′ to 3′, homology region 1, a splice acceptor (SA) to splice to AAVS1 exon 1, a coding sequence for self-cleaving peptide 2A, a coding sequence for a puromycin-resistance gene, a polyA signal sequence, a 5′ genomic insulator sequence, the doxycycline-inducible commitment factor cassette, the rtTA coding sequence driven off a CAGG promoter and followed by a polyA sequence, a 3′ genomic insulator sequence, and homology region 2. The genomic insulator sequences ensure the transgenes within them are not epigenetically silenced over the course of differentiation. The homology regions are homologous to the genomic regions flanking the ZFN cleavage site. Cells with successful targeted integration (TI) can be positively selected for by introducing puromycin into culture. Inducible expression of the Treg induction factors is useful since certain factors may be toxic during mesodermal, hematopoietic, or lymphocyte development, thus turning on the factors only during T cell development to skew differentiation towards the Treg lineage is advantageous.

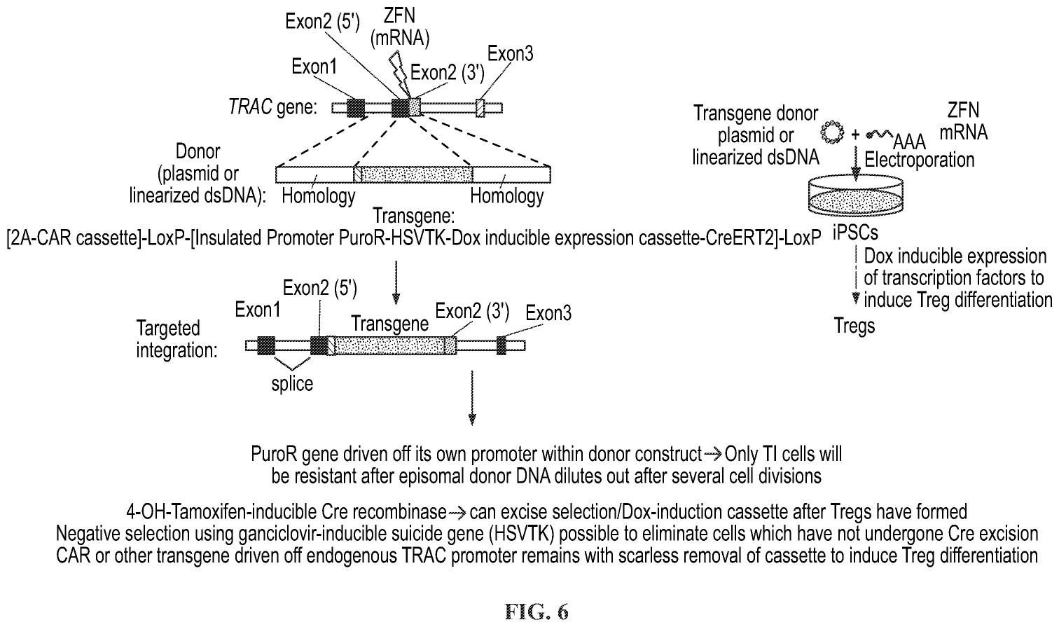

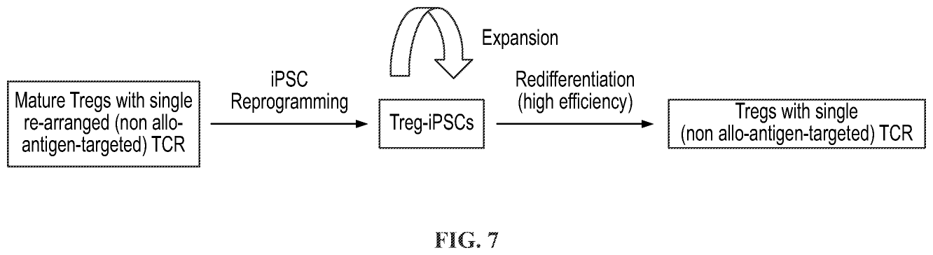

In some embodiments, the heterologous sequence contains an expression cassette for an antigen-binding receptor, such as a chimeric antigen receptor (CAR). FIGS. 5 and 6 illustrate examples of such embodiments. In FIG. 5, the heterologous sequence is introduced by plasmid DNA or linearized double-stranded DNA and contains, from 5′ to 3′, homology region 1, a CAR expression cassette (in antisense orientation to the donor) driven off its own promoter and containing a polyA site, a 5′ LoxP site, a splice acceptor to splice to AAVS1 exon 1, a coding sequence for self-cleaving peptide 2A, a coding sequence for a puromycin-resistance gene, a coding sequence for the suicide gene HSV-TK, a polyA site, a 5′ genomic insulator sequence, the doxycycline-inducible commitment factor expression cassette, the rtTA coding sequence driven off a CAGG promoter, the coding sequence for a 4-hydroxy-tamoxifen (4-OHT)-inducible form of the Cre recombinase linked to the rtTA sequence via a 2A peptide and followed by a polyA sequence, a 3′ genomic insulator sequence, a 3′ LoxP site, and homology region 2. The genomic insulator sequences ensure the transgenes within them are not epigenetically silenced over the course of differentiation. The homology regions are homologous to the genomic regions flanking the ZFN cleavage site. Cells with successful targeted integration can be positively selected for by introducing puromycin into the tissue culture. The constitutively expressed 4-OHT-inducible Cre allows for excision of the entire cassette between the LoxP sites after addition of 4-OHT to culture. Cells that have not undergone recombinase-mediated excision will still express HSV-TK and thus can be negatively selected (eliminated) by adding ganciclovir (GCV) into tissue culture. GCV will result in cell death of any cell expressing HSV-TK. This system allows for completely scarless removal of the Treg induction cassette while leaving the CAR cassette integrated to allow for targeted immunosuppression in the engineered Tregs.

FIG. 6 illustrates expression of CAR from the engineered TRAC gene. In this example, the heterologous sequence, introduced by plasmid DNA or linearized dsDNA, contains, from 5′ to 3′, homology region 1, a 2A-coding sequence fused directly to the CAR coding sequence followed by a polyA site, a 5′ LoxP site, a 5′ genomic insulator sequence, a splice acceptor to splice to AAVS1 exon 1, a 2A coding sequence, a coding sequence for a puromycin-resistance gene with a 2A peptide-linked coding sequence for the suicide gene HSV-TK, both driven off their own promoter and followed by a polyA signal sequence, the doxycycline-inducible Treg induction factor expression cassette, the rtTA coding sequence driven off a CAGG promoter, the coding sequence for a 4-OHT-inducible form of the Cre recombinase linked to the rtTA sequence via a 2A peptide and followed by a polyA sequence, a 3′ genomic insulator sequence, a 3′ LoxP site, and homology region 2. The genomic insulator sequences ensure the transgenes within them are not epigenetically silenced over the course of differentiation. The homology regions are homologous to the genomic regions flanking the ZFN cleavage site. Cells with successful targeted integration can be positively selected for by introducing puromycin into tissue culture (optionally waiting a week or more for unintegrated donor episomes to dilute out). The constitutively expressed 4-OHT-inducible Cre allows for excision of the entire cassette between the LoxP sites after addition of 4-OHT to culture. Cells which have not undergone recombinase-mediated excision will still express HSV-TK and thus can be eliminated by adding GCV into the tissue culture. This system allows for completely scarless removal of the Treg induction cassette while leaving the CAR cassette driven off the endogenous TRAC promoter integrated to allow for targeted immunosuppression in the engineered Tregs.

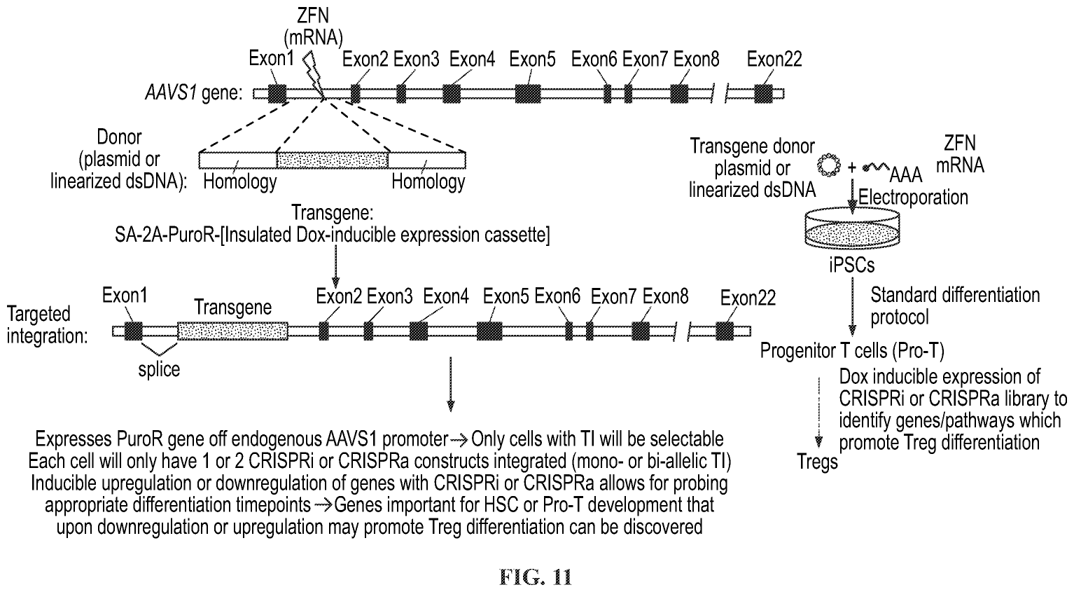

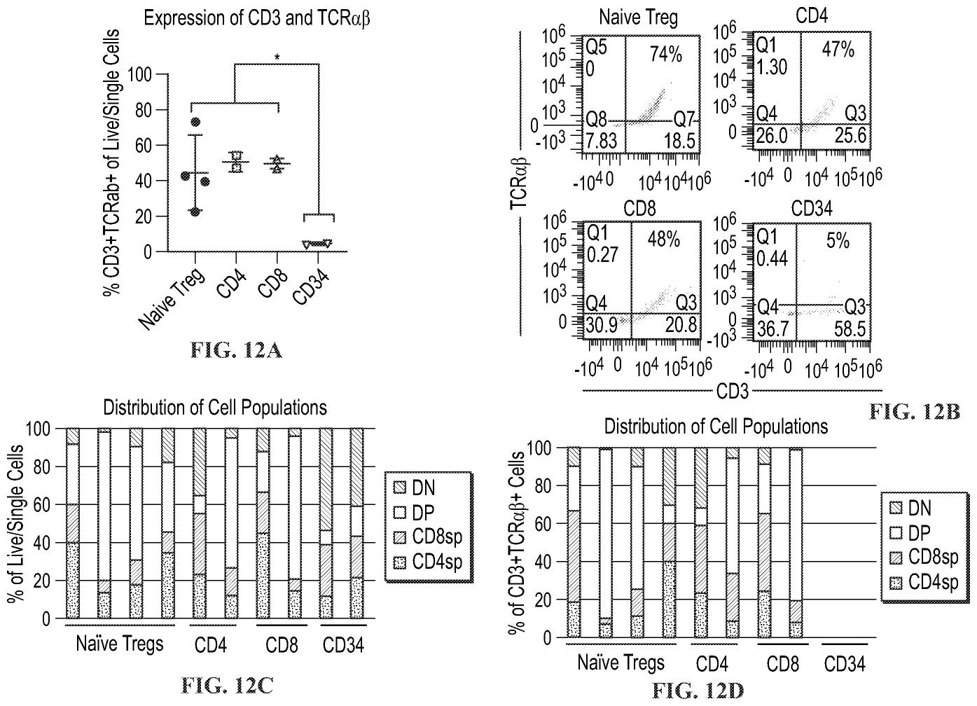

FIG. 11 is a schematic diagram depicting a genome editing approach to integrating either a CRISPR activation (CRISPRa) or inihibition (CRISPRi) library, which includes either a dead Cas9 (dCas9) fused to either the VPH activating domain or KRAB inhibition domain, respectively, driven off a doxycycline-inducible promoter into intron 1 of the human AAVS1 gene. Upon the introduction of doxycycline to culture, the constitutively expressed inducible form of the tetracycline-controlled transactivator (rtTA) binds to the Tet-responsive element and initiates transcription of the integrated CRISPRa or CRISPRi constructs. These libraries contain gRNAs targeted to every coding gene in the human genome, whereas only one or two dCas9-gRNA constructs at maximum (mono- or b-allelic targeted integration) will be integrated per cell. A ZFN produced from an introduced mRNA makes a double-stranded break at a specific site (lightning bolt) in intron 1. The donor sequence, introduced by plasmid DNA or linearized dsDNA, contains, from 5′ to 3′, homology region 1, a splice acceptor to splice to AAVS1 exon 1, a coding sequence for self-cleaving peptide 2A, a coding sequence for a puromycin-resistance gene, a polyA signal sequence, a 5′ genomic insulator sequence, the doxycycline-inducible CRISPRa or CRISPRi construct library, the rtTA coding sequence driven off a CAGG promoter and followed by a polyA sequence, a 3′ genomic insulator sequence, and homology region 2. The genomic insulator sequences ensure the transgenes within them are not epigenetically silenced over the course of differentiation. The homology regions are homologous to the genomic regions flanking the ZFN cleavage site. Cells with successful targeted integration can be positively selected for by introducing puromycin into culture. Inducible expression of the CRISPRa or CRISPRi construct is useful since upregulation or downregulation of certain genes targeted within the libraries may be toxic during mesodermal, hematopoietic, or lymphocyte development, thus turning on or off the factors only during T cell development (after the progenitor T cell stage) to skew differentiation towards the Treg lineage is advantageous and may allow for novel Treg induction factor and pathways to be discovered.

The above-described figures are merely illustrative of some embodiments of the present invention. For example, other self-cleaving peptides may be used in lieu of the T2A and P2A peptides illustrated in the figures. Self-cleaving peptides are viral derived peptides with a typical length of 18-22 amino acids. Self-cleaving 2A peptides include T2A, P2A, E2A, and F2A. Moreover, codon diversified versions of the 2A peptides may be used to combine multiple Treg induction genes on one large integrated transgene cassette. In some embodiments, IRES is used in in lieu of a self-cleaving peptide coding sequence. Both introns and exons may be targeted. Additional elements may be included in the heterologous sequence. For example, the heterologous sequence may include RNA-stabilizing elements such as a Woodchuck Hepatitis Virus Posttranscriptional Regulatory Element (WPRE).

### III. Gene Editing Methods

Any gene editing method for targeted integration of a heterologous sequence into a specific genomic site may be used. To enhance the precision of site-specific integration of the transgene, a construct carrying the heterologous sequence may contain on either or both of its ends a homology region that is homologous to the targeted genomic site. In some embodiments, the heterologous sequence carries in both of 5′ and 3′ end regions sequences that are homologous to the target genomic site in a T cell specific gene locus or a genomic safe harbor gene locus. The lengths of the homology regions on the heterologous sequence may be, for example, 50-1,000 base pairs in length. The homology region in the heterologous sequence can be, but need not be, identical to the targeted genomic sequence. For example, the homology region in the heterologous sequence may be at 80 or more percent (e.g., 85 or more, 90 or more, 95 or more, 99 or more percent) homologous or identical to the targeted genomic sequence (e.g., the sequence that is to be replaced by the homology region in the heterologous sequence). In further embodiments, the construct, when linearized, comprise on one end homology region 1, and on its other end homology region 2, where homology regions 1 and 2 are respectively homologous to genomic region 1 and genomic region 2 flanking the integration site in the genome.

The construct carrying the heterologous sequence can be introduced to the target cell by any known techniques such as chemical methods (e.g., calcium phosphate transfection and lipofection), non-chemical methods (e.g., electroporation and cell squeezing), particle-based methods (e.g., magnetofection), and viral transduction (e.g., by using viral vectors such as vaccinia vectors, adenoviral vectors, lentiviral vectors, adeno-associated viral (AAV) vectors, retroviral vectors, and hybrid viral vectors). In some embodiments, the construct is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system). The AAV may be of any serotype, for example, AAV1, AAV2, AAV3, AAV4, AAV5, AAV6, AAV7, AAV8, AAV8.2, AAV9, or AAVrh10, of a pseudotype such as AAV2/8, AAV2/5, or AAV2/6.

The heterologous sequence may be integrated to the TRAC genomic locus by any site-specific gene knockin technique. Such techniques include, without limitation, homologous recombination, gene editing techniques based on zinc finger nucleases or nickases (collectively “ZFNs” herein), transcription activator-like effector nucleases or nickases (collectively “TALENs” herein), clustered regularly interspaced short palindromic repeat systems (CRISPR, such as those using Cas9 or cpf1), meganucleases, integrases, recombinases, and transposes. As illustrated below in the Working Examples, for site-specific gene editing, the editing nuclease typically generates a DNA break (e.g., a single- or double-stranded DNA break) in the targeted genomic sequence such that a donor polynucleotide having homology to the targeted genomic sequence (e.g., the construct described herein) is used as a template for repair of the DNA break, resulting in the introduction of the donor polynucleotide to the genomic site.

Gene editing techniques are well known in the art. See, e.g., U.S. Pat. Nos. 8,697,359, 8,771,945, 8,795,965, 8,865,406, 8,871,445, 8,889,356, 8,895,308, 8,906,616, 8,932,814, 8,945,839, 8,993,233, 8,999,641, 9,790,490, 10,000,772, 10,113,167, and 10,113,167 for CRISPR gene editing techniques. See, e.g., U.S. Pat. Nos. 8,735,153, 8,771,985, 8,772,008, 8,772,453, 8,921,112, 8,936,936, 8,945,868, 8,956,828, 9,234,187, 9,234,188, 9,238,803, 9,394,545, 9,428,756, 9,567,609, 9,597,357, 9,616,090, 9,717,759, 9,757,420, 9,765,360, 9,834,787, 9,957,526, 10,072,062, 10,081,661, 10,117,899, 10,155,011, and 10,260,062 for ZFN techniques and its applications in editing T cells and stem cells. The disclosures of the aforementioned patents are incorporated by reference herein in their entirety.

In gene editing techniques, the gene editing complex can be tailored to target specific genomic sites by altering the complex's DNA binding specificity. For example, in CRISPR technology, the guide RNA sequence can be designed to bind a specific genomic region; and in the ZFN technology, the zinc finger protein domain of the ZFN can be designed to have zinc fingers specific for a specific genomic region, such that the nuclease or nickase domains of the ZFN can cleave the genomic DNA at a site-specific manner. Depending on the desired genomic target site, the gene editing complex can be designed accordingly.

Components of the gene editing complexes may be delivered into the target cells, concurrent with or sequential to the transgene construct, by well-known methods such as electroporation, lipofection, microinjection, biolistics, virosomes, liposomes, lipid nanoparticles, immunoliposomes, polycation or lipid:nucleic acid conjugates, naked DNA or mRNA, and artificial virions. Sonoporation using, e.g., the Sonitron 2000 system (Rich-Mar) can also be used for delivery of nucleic acids. In particular embodiments, one or more components of the gene editing complex, including the nuclease or nickase, are delivered as mRNA into the cells to be edited.

### IV. Antigen-Specificity of the Tregs

In some embodiments, the stem cells or progenitor cells may be further engineered (e.g., using gene editing methods described herein) to include transgenes encoding an antigen-recognition receptor such as a TCR or a CAR. Alternatively, the stem cells or progenitor cells are cells that have been reprogrammed from mature Tregs that have already rearranged their TCR alpha/beta (or delta/gamma) loci, and Tregs re-differentiated from such stem or progenitor cells will retain the antigen specificity of their ancestral Tregs. In any event, the Tregs may be selected for their specificity for an antigen of interest for a particular therapeutic goal.

In some embodiments, the antigen of interest is a polymorphic allogeneic MHC molecule, such as one expressed by cells in a solid organ transplant or by cells in a cell-based therapy (e.g., bone marrow transplant, cancer CAR T therapy, or cell-based regenerative therapy). MHC molecules so targeted include, without limitation, HLA-A, HLA-B, or HLA-C; HLA-DP, HLA-DM, HLA-DOA, HLA-DOB, HLA-DQ, or HLA-DR. By way of example, the antigen of interest is class I molecule HLA-A2. HLA-A2 is a commonly mismatched histocompatibility antigen in transplantation. HLA-A mismatching is associated with poor outcomes after transplantation. Engineered Tregs expressing a CAR specific for an MHC class I molecule are advantageous because MHC class I molecules are broadly expressed on all tissues, so the Tregs can be used for organ transplantation regardless of the tissue type of the transplant. Tregs against HLA-A2 offers the additional advantage that HLA-A2 is expressed by a substantial proportion of the human population and therefore on many donor organs. There has been evidence showing that expression of an HLA-A2 CAR in Treg cells can enhance the potency of the Treg cells in preventing transplant rejection (see, e.g., Boardman, supra; MacDonald et al., J Cin Invest. (2016) 126(4):1413-24; and Dawson, supra).

In some embodiments, the antigen of interest is an autoantigen, i.e., an endogenous antigen expressed prevalently or uniquely at the site of autoimmune inflammation in a specific tissue of the body. Tregs specific for such an antigen can home to the inflamed tissue and exert tissue-specific activity by causing local immunosuppression. Examples of autoantigens are aquaporin water channels (e.g., aquaporin-4 water channel), paraneoplastic antigen Ma2, amphiphysin, voltage-gated potassium channel, N-methyl-d-aspartate receptor (NMDAR), a-amino-3-hydroxy-5-methyl-4-isoxazoleproprionic acid receptor (AMPAR), thyroid peroxidase, thyroglobulin, anti-N-methyl-D-aspartate receptor (NR1 subunit), Rh blood group antigens, desmoglein 1 or 3 (Dsg1/3), BP180, BP230, acetylcholine nicotinic postsynaptic receptors, thyrotropin receptors, platelet integrin, glycoprotein IIb/IIIa, calpastatin, citrullinated proteins, alpha-beta-crystallin, intrinsic factor of gastric parietal cells, phospholipase A2 receptor 1 (PLA2R1), and thrombospondin type 1 domain-containing 7A (THSD7A). Additional examples of autoantigens are multiple sclerosis-associated antigens (e.g., myelin basic protein (MBP), myelin associated glycoprotein (MAG), myelin oligodendrocyte glycoprotein (MOG), proteolipid protein (PLP), oligodendrocyte myelin oligoprotein (OMGP), myelin associated oligodendrocyte basic protein (MOBP), oligodendrocyte specific protein (OSP/Claudin 11), oligodendrocyte specific proteins (OSP), myelin-associated neurite outgrowth inhibitor NOGO A, glycoprotein Po, peripheral myelin protein 22 (PMP22), 2′3′-cyclic nucleotide 3′-phosphodiesterase (CNPase), and fragments thereof); joint-associated antigens (e.g., citrulline-substituted cyclic and linear filaggrin peptides, type II collagen peptides, human cartilage glycoprotein 39 peptides, keratin, vimentin, fibrinogen, and type I, III, IV, and V collagen peptides); and eye-associated antigens (e.g., retinal arrestin, S-arrestin, interphotoreceptor retinoid-binding proteins, beta-crystallin B1, retinal proteins, choroid proteins, and fragments thereof). In some embodiments, the autoantigen targeted by the Treg cells is IL23-R (for treatment of, e.g., Crohn's disease, inflammatory bowel disease, or rheumatoid arthritis), MOG (for treatment of multiple sclerosis), or MBP (for treatment of multiple sclerosis). In some embodiments, the Tregs may target other antigens of interest (e.g., B cell markers CD19 and CD20).

In some embodiments, Tregs recognizing foreign peptides (e.g., CMV, EBV, and HSV), rather than allo-antigens, can be used in an allogeneic adoptive cell transfer setting without the risk of being constantly activated by recognizing allo-antigens without the need for knockout out of TCR expression.

### V. Cells Used for Genome Editing

The engineered cells of the present disclosure are mammalian cells, such as human cells, cells from a farm animal (e.g., a cow, a pig, or a horse), and cells from a pet (e.g., a cat or a dog). The source cells, i.e., cells on which the genome editing is performed, may be pluripotent stem cells (PSCs). PSCs are cells capable to giving rise to any cell type in the body and include, for example, embryonic stem cells (ESCs), PSCs derived by somatic cell nuclear transfer, and induced PSCs (iPSCs). See, e.g., Iriguchi and Kaneko, Cancer Sci. (2019) 110(1):16-22 for differentiating iPSCs to T cells. As used herein, the term “embryonic stem cells” refers to pluripotent stem cells obtained from early embryos; in some embodiments, this term refers to ESCs obtained from a previously established embryonic stem cell line and excludes stem cells obtained by recent destruction of a human embryo.

In other embodiments, the source cells for genome editing are multipotent cells such as mesodermal stem cells, mesenchymal stem cells, hematopoietic stem cells (e.g., those isolated from bone marrow or cord blood), or hematopoietic progenitor cells (e.g., lymphoid progenitor cells). Multipotent cells are capable of developing into more than one cell type, but are more limited in cell type potential than pluripotent cells. The multipotent cells may be derived from established cell lines or isolated from human bone marrow or umbilical cords. By way of example, the hematopoietic stem cells (HSC) may be isolated from a patient or a healthy donor following granulocyte-colony stimulating factor (G-CSF)-induced mobilization, plerixafor-induced mobilization, or a combination thereof. To isolate HSCs from the blood or bone marrow, the cells in the blood or bone marrow may be panned by antibodies that bind unwanted cells, such as antibodies to CD4 and CD8 (T cells), CD45 (B cells), GR-1 (granulocytes), and lad (differentiated antigen-presenting cells) (see, e.g., Inaba, et al. (1992) J. Exp. Med. 176:1693-1702). HSCs can then be positively selected by antibodies to CD34.

In some embodiments, the cells to be engineered are iPSCs reprogrammed from a mature Treg (Takahashi et al. (2007) Cell 131(5):861-72), such as a mature Treg expressing a TCR that targets a non-allogenic antigen. See FIG. 7 and further discussions below.

The edited stem cells and/or progenitor cells may be differentiated into Treg cells in vitro before engrafting into a patient, as further discussed below. Alternatively, the stem and/or edited progenitor cells may be induced to differentiate into Treg cells after engrafting to a patient.

1. Additional Genome Editing

The present engineered cells may be further genetically engineered, before or after the genome editing described above, to make the cells more effective, more useable on a larger patient population, and/or safer. The genetic engineering may be done by, e.g., random insertion of a heterologous sequence of interest (e.g., by using a lentiviral vector, a retroviral vector, or a transposon) or targeted genomic integration (e.g., by using genome editing mediated by ZFN, TALEN, CRISPR, site-specific engineered recombinase, or meganuclease).

For example, the cells may be engineered to express one or more exogenous CAR or TCR through a site-specific integration of a CAR or TCR transgene into the genome of the cell. The exogenous CAR or TCR may target an antigen of interest, as described above.

The cells may also be edited to encode one or more therapeutic agents to promote the immunosuppressive activity of the Tregs. Examples of therapeutic agents include cytokines (e.g., IL-10), chemokines (e.g., CCR7), growth factors (e.g., remyelination factors for treatment of multiple sclerosis), and signaling factors (e.g., amphiregulin).

In additional embodiments, the cells are further engineered to express a factor that reduces severe side effects and/or toxicities of cell therapy, such as cytokine release syndrome (CRS) and/or neurotoxicities (e.g., an anti-IL-6 scFv or a secretable IL-12) (see, e.g., Chmielewski et al., Immunol Rev. (2014) 257(1):83-90).

In some embodiments, EZH1 signaling is disrupted in the engineered cells to enhance their lymphoid commitment (see, e.g., Vo et al., Nature (2018) 553(7689):506-10).

In some embodiments, the edited cells may be allogeneic cells to the patient. In such instances, the cells may be further engineered to reduce host rejection to these cells (graft rejection) and/or these cells' potential attack on the host (graft-versus-host disease). The further-engineered allogeneic cells are particularly useful because they can be used in multiple patients without compatibility issues. The allogeneic cells thus can be called “universal” and can be used “off the shelf” The use of “universal” cells greatly improves the efficiency and reduces the costs of adopted cell therapy.

To generate “universal” allogeneic cells, the cells may be engineered, for example, to have a null genotype for one or more of the following: (i) T cell receptor (TCR alpha chain or beta chain); (ii) a polymorphic major histocompatibility complex (MHC) class I or II molecule (e.g., HLA-A, HLA-B, or HLA-C; HLA-DP, HLA-DM, HLA-DOA, HLA-DOB, HLA-DQ, or HLA-DR; or β2-microglobulin (B2M)); (iii) a transporter associated with antigen processing (e.g., TAP-1 or TAP-2); (iv) Class II MHC transactivator (CIITA); (v) a minor histocompatibility antigen (MiHA; e.g., HA-1/A2, HA-2, HA-3, HA-8, HB-1H, or HB-1Y); (vi) immune checkpoint inhibitors such as PD-1 and CTLA-4; (vii) VIM; and (vi) any combination thereof.

The allogeneic engineered cells may also express an invariant HLA or CD47 to increase the resistance of the engineered cells (especially those with HLA class I knockout or knockdown) to the host's natural killer and other immune cells involved in anti-graft rejection. For example, the heterologous sequence carrying the commitment factor transgene may additionally comprise a coding sequence for an invariant HLA (e.g., HLA-G, HLA-E, and HLA-F) or CD47. The invariant HLA or CD47 coding sequence may be linked to the primary transgene in the heterologous sequence through a coding sequence for a self-cleaving peptide or an IRES sequence.

2. Safety Switch in Engineered Cells

In cell therapy, it may be desirable for the transplanted cells to contain a “safety switch” in their genomes, such that proliferation of the cells can be stopped when their presence in the patient is no longer desired (see, e.g., Hartmann et al., EMBO Mol Med. (2017) 9:1183-97). A safety switch may, for example, be a suicide gene, which upon administration of a pharmaceutical compound to the patient, will be activated or inactivated such that the cells enter apoptosis. A suicide gene may encode an enzyme not found in humans (e.g., a bacterial or viral enzyme) that converts a harmless substance into a toxic metabolite in the human cell.

In some embodiments, the suicide gene may be a thymidine kinase (TK) gene from Herpes Simplex Virus (HSV). TK can metabolize ganciclovir, valganciclovir, famciclovir, or another similar antiviral drug into a toxic compound that interferes with DNA replication and results in cell apoptosis. Thus, a HSV-TK gene in a host cell can be turned on to kill the cell by administration of one of such antiviral drugs to the patient.

In other embodiments, the suicide gene encodes, for example, another thymidine kinase, a cytosine deaminase (or uracil phosphoribosyltransferase; which transforms anti-fungal drug 5-fluorocytosine into 5-fluorouracil), a nitroreductase (which transforms CB1954 (for [5-(aziridin-1-yl)-2,4-dinitrobenzamide]) into a toxic compound), 4-hydroxylamine), and a cytochrome P450 (which transforms ifosfamide to acrolein (nitrogen mustard)) (Rouanet et al., Int J Mol Sci. (2017) 18(6):E1231), or inducible caspase-9 (Jones et al., Front Pharmacol. (2014) 5:254). In additional embodiments, the suicide gene may encode an intracellular antibody, a telomerase, another caspase, or a DNAase. See, e.g., Zarogoulidis et al., J Genet Syndr Gene Ther. (2013) doi:10.4172/2157-7412.1000139.

A safety switch may also be an “on” or “accelerator” switch, a gene encoding a small interfering RNA, an shRNA, or an antisense that interferences the expression of a cellular protein critical for cell survival.

The safety switch may utilize any suitable mammalian and other necessary transcription regulatory sequences. The safety switch can be introduced into the cell through random integration or site-specific integration using gene editing techniques described herein or other techniques known in the art. It may be desirable to integrate the safety switch in a genomic safe harbor such that the genetic stability and the clinical safety of the engineered cell are maintained. Examples of safe harbors as used in the present disclosure are the AAVS1 locus; the ROSA26 locus; the CLYBL locus; the gene loci for albumin, CCR5, and CXCR4; and the locus where the endogenous gene is knocked out in the engineered cells (e.g., the T cell receptor alpha or beta chain gene locus, the HLA gene locus, the CIITA locus, or the β2-microglobulin gene locus).

### VI. Reprogramming and Differentiating Cells In Vitro

The cells of the present disclosure can be reprogrammed from mature Treg cells and/or differentiated into Treg cells in tissue culture using methods known in the art. The methods described below are merely illustrative and are not limiting.

1. Reprogramming Treg Cells into iPSCs

In certain embodiments, the source cells for genetic engineering are induced pluripotent stem cells reprogrammed from an adult, adolescent, or fetal Treg cell (Takahashi et al., Cell (2007) 131(5):861-72). In these embodiments, the reprogrammed stem cell would retain the epigenetic memory of its original Treg phenotype (Kim et al., Nature (2010) 467(7313):285-90) and thus may re-differentiate back into a Treg with higher efficiency than other stem cells such as those reprogrammed from a different cell type. A stem cell reprogrammed from a Treg would also retain the V(D)J-rearranged TCR loci, which may further enhance the Treg differentiation potential of the stem cell because V(D)J recombination is a development hurdle during T cell ontogeny (see, e.g., Nishimura et al., Cell Stem Cell (2013) 12(1):114-26).

The Treg cells to be used for reprogramming may be isolated from a number of sources, including peripheral blood mononuclear cells (PBMC), bone marrow, lymph node tissue, cord blood, thymus tissue, or spleen tissue. For example, Tregs may be isolated from a unit of blood collected from a subject using well known techniques such as Ficoll™ separation, centrifugation through a PERCOLL™ gradient following red blood cell lysis and monocyte depletion, counterflow centrifugal elutriation, leukapheresis, and subsequent cell surface marker-based magnetic or flow cytometric isolation.

Further enrichment of Treg cells from the isolated white blood cells can be accomplished by positive and/or negative selection with a combination of antibodies directed to unique surface markers using techniques such as flow cytometry cell sorting and/or magnetic immunoadherence involving conjugated beads. For example, to enrich for CD4⁺cells by negative selection, a monoclonal antibody cocktail typically may include antibodies to CD14, CD20, CD11b, CD16, HLA-DR, and CD8. To enrich or positively select for Tregs, antibodies to CD4, CD25, CD45RA, CD62L, GITR, and/or CD127 can be used.

In an exemplary and nonlimiting protocol, Treg cells may be obtained as follows (see Dawson et al., JCI Insight. (2019) 4(6):e123672). CD4⁺ T cells are isolated from a human donor via RosetteSep (STEMCELL Technologies, 15062) and enriched for CD25⁺ cells (Miltenyi Biotec, 130-092-983) prior to sorting live CD4⁺CD25iCD127^{lo} Tregs or CD4⁺CD127^{lo} CD25^{hi}CD45RA⁺ Tregs using a MoFlo Astrios (Beckman Coulter) or FACS Aria II (BD Biosciences). Sorted Tregs may be stimulated with L cells and anti-CD3 monoclonal antibody (e.g., OKT3, UBC AbLab; 100 ng/ml) in ImmunoCult-XF T cell expansion media (STEMCELL Technologies,10981) with 1000 U/ml IL-2 (Proleukin) as described in MacDonald et al., J Cin Invest. (2016) 126(4):1413-24). One or more days later, the Treg cells may be reprogrammed (de-differentiated) into stem cells as described below. For phenotypic analysis, cells may be stained with fixable viability dye (FVD, Thermo Fisher Scientific, 65-0865-14; BioLegend, 423102) and for surface markers before fixation and permeabilization using an eBioscience FOXP3/Transcription Factor Staining Buffer Set (Thermo Fisher Scientific, 00-5523-00) and staining for intracellular proteins. Samples were read on a CytoFLEX (Beckman Coulter).

The Tregs can then be reprogrammed into iPSCs using reprogramming factors such as OCT3/4, SOX2, KLF4, and c-MYC (or L-MYC) (see, e.g., Nishino et al., Regen Ther. (2018) 9:71-8). Reprogramming factors may be delivered via non-integrating methods (e.g., Sendai virus, plasmid, RNA, minicircle, AAV, IDLV, etc.) or integrating methods (e.g., lentivirus, retrovirus, and nuclease-mediated targeted integration).

FIG. 7 illustrates the process of reprogramming mature Tregs into iPSCs, which are then expanded, and re-differentiated at high efficiency to Treg cells. This process provides an expanded, “rejuvenated” pool of Treg cells from a single Treg cell.

2. Skewing Differentiation of Stem Cells Towards CD4⁺ Treg Lineage

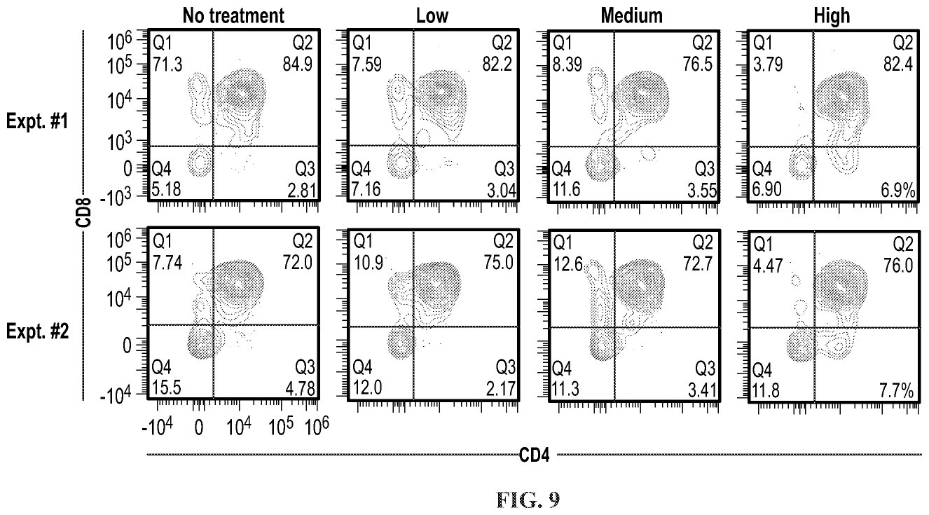

The engineered stem cells have increased Treg-differentiation potential due to the presence of commitment factor-encoding transgenes in their genome. FIG. 8 illustrates a stepwise differentiation process in which an iPSC differentiates into a Treg cell: iPSC, mesodermal stem (progenitor) cell, HSC, lymphoid progenitor cell, progenitor T cell, immature single positive (CD4⁺ or CD8⁺) T cell, double positive T cell (CD4⁺CD8⁺), mature CD4⁺ T cell, and finally Treg cell. To skew the differentiation of these stem cells toward becoming CD4⁺ T cells and ultimately Treg cells, tissue culture techniques can be employed.

In some embodiments, the stem cells are subjected to IL-7Ra (CD127) signaling blockade during the later stages of T cell development to skew differentiation into CD4⁺ T cell and Treg lineages (see Singer et al., Nat Rev Immunol. (2008) 8(10):788-801; FIG. 8 and FIG. 9). In other embodiments, CCR7 signaling is blocked during T cell development. CCR7 has been shown to be upregulated in CD8⁺ T cells as compared to CD4⁺ T cells and to promote the commitment of progenitor T cells to the CD8⁺ fate (see Yin et al., J Immunol. (2007) 179(11):7358-64). In certain embodiments, IL-2 concentrations are lowered to provide a proliferative growth advantage to Tregs, which express high levels of high affinity IL-2 receptor (CD25) (Singer, supra; FIG. 8). In certain embodiments, activation beads that preferentially promote Treg proliferation are used to activate and expand Tregs preferentially compared to effector T cells (e.g. Treg Xpander beads from Thermo Fisher Scientific).

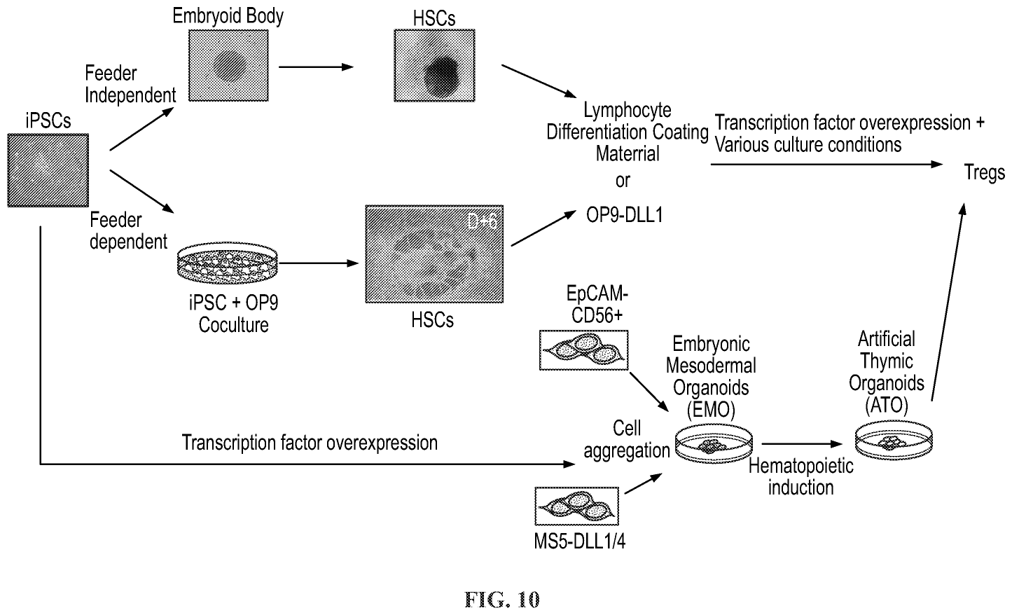

FIG. 10 illustrates additional tissue culture techniques that can be employed. In some embodiments illustrated therein, the engineered stem cells are co-cultured with mesenchymal stromal cells (see Di Ianni et al., Exp Hematol. (2008) 36(3):309-18). Examples of such stromal cells include OP9 or OP9-DLL1 stromal cells, which promote lymphoid commitment (see Hutton et al., J Leukocyte Biology (2009) 85(3):445-51; FIG. 10). In other embodiments, embryonic mesodermal progenitors are formed from pluripotent stem cells and are cultured in three-dimensional embryonic mesodermal organoids via co-culture on MS5-DLL1/4 cells or EpCAM⁻CD56⁺ stromal cells (FIG. 10). These embryonic mesodermal progenitors are then differentiated into artificial thymic organoids to more accurately replicate the process of thymic development (Montel-Hagen et al., Cell Stem Cell (2019) 24(3):376-89.e8; Seet et al., Nat Methods (2017) 14(5): 521-30).

3. Maintenance of Treg Phenotype

Plasticity is a property inherent to nearly all types of immune cells. It appears that Treg cells are able to transition (“drift”) to Teff cells under inflammatory and environmental conditions (see Sadlon et al., Clin Transl Immunol. (2018) 7(2):e1011). To maintain the Treg phenotype and/or to increase expression of the transgene(s) (e.g., FOXP3, Helios, and/or ThPOK) in the engineered Treg cells, the cells may be cultured in tissue culture media containing rapamycin and/or a high concentration of IL-2 (see, e.g., MacDonald et al., Clin Exp Immunol. (2019) doi: 10.1111/cei.13297). In some embodiments, to preferentially expand Tregs compared to Teff, the cells may be cultured in tissue culture media containing low-dose IL-2 (see, e.g., Congxiu et al., Signal Transduct Targ Ther. (2018) 3(2):1-10).

### VII. Use of the Engineered Treg Cells

The genetically engineered Treg cells of the present disclosure can be used in cell therapy to treat a patient (e.g., a human patient) in need of induction of immune tolerance or restoration of immune homeostasis. The terms “treating” and “treatment” refer to alleviation or elimination of one or more symptoms of the treated condition, prevention of the occurrence or reoccurrence of the symptoms, reversal or remediation of tissue damage, and/or slowing of disease progression.

A patient herein may be one having or at risk of having an undesired inflammatory condition such as an autoimmune disease. Examples of autoimmune diseases are Addison's disease, AIDS, ankylosing spondylitis, anti-glomerular basement membrane disease autoimmune hepatitis, dermatitis, Goodpasture's syndrome, granulomatosis with polyangiitis, Graves' disease, Guillain-Barre syndrome, Hashimoto's thyroiditis, hemolytic anemia, Henoch-Schonlein purpura (HSP), juvenile arthritis, juvenile myositis, Kawasaki disease, inflammatory bowel diseases (such as Crohn's disease and ulcerative colitis), polymyositis, pulmonary alveolar proteinosis, multiple sclerosis, myasthenia gravis, neuromyelitis optica, PANDAS, psoriasis, psoriatic arthritis, rheumatoid arthritis, Sjögren's syndrome, systemic scleroderma, systemic sclerosis, systemic lupus erythematosus, thrombocytopenic purpura (TTP), Type I diabetes mellitus, uveitis, vasculitis, vitiligo, and Vogt-Koyanagi-Harada Disease.

In some embodiments, the Tregs express an antigen-binding receptor (e.g., TCR or CAR) targeting an autoantigen associated with an autoimmune disease, such as myelin oligodendrocyte glycoprotein (multiple sclerosis), myelin protein zero (autoimmune peripheral neuropathy), HIV env or gag protein (AIDS), myelin basic protein (multiple sclerosis), CD37 (systemic lupus erythematosus), CD20 (B-cell mediated autoimmune diseases), and IL-23R (inflammatory bowel diseases such as Crohn's disease or ulcerative colitis).

A patient herein may be one in need an allogeneic transplant, such as an allogeneic tissue or solid organ transplant or an allogeneic cell therapy. The Tregs of the present disclosure, such as those expressing CARs targeting one or more allogeneic MHC class I or II molecules, may be introduced to the patient, where the Tregs will home to the transplant and suppress allograft rejection elicited by the host immune system and/or graft-versus-host rejection. Patient in need of a tissue or organ transplant or an allogeneic cell therapy include those in need of, for example, kidney transplant, heart transplant, liver transplant, pancreas transplant, intestine transplant, vein transplant, bone marrow transplant, and skin graft; those in need of regenerative cell therapy; those in need of gene therapy (AAV-based gene therapy); and those in need in need of cancer CAR T therapy.

If desired, the patient receiving the engineered Tregs herein (which includes patients receiving engineered pluripotent or multipotent cells that will differentiate into Tregs in vivo) is treated with a mild myeloablative procedure prior to introduction of the cell graft or with a vigorous myeloablative conditioning regimen.

The engineered cells of the present disclosure may be provided in a pharmaceutical composition containing the cells and a pharmaceutically acceptable carrier. For example, the pharmaceutical composition comprises sterilized water, physiological saline or neutral buffered saline (e.g., phosphate-buffered saline), salts, antibiotics, isotonic agents, and other excipients (e.g., glucose, mannose, sucrose, dextrans, mannitol; proteins (e.g., human serum albumin); amino acids (e.g., glycine and arginine); antioxidants (e.g., glutathione); chelating agents (e.g., EDTA); and preservatives). The pharmaceutical composition may additionally comprise factors that are supportive of the Treg phenotype and growth (e.g., IL-2 and rapamycin or derivatives thereof), anti-inflammatory cytokines (e.g., IL-10, TGF-β, and IL-35), and other cells for cell therapy (e.g., CAR T effector cells for cancer therapy or cells for regenerative therapy). For storage and transportation, the cells optionally may be cryopreserved. Prior to use, the cells may be thawed and diluted in a pharmaceutically acceptable carrier.

The pharmaceutical composition of the present disclosure is administered to a patient in a therapeutically effective amount through systemic administration (e.g., through intravenous injection or infusion) or local injection or infusion to the tissue of interest (e.g., infusion through the hepatic artery, and injection to the brain, heart, or muscle). The term “therapeutically effective amount” refers to the amount of a pharmaceutical composition, or the number of cells, that when administered to the patient, is sufficient to effect the treatment.

In some embodiments, a single dosing unit of the pharmaceutical composition comprises more than 10⁴ cells (e.g., from about 105 to about 10⁶ cells, from about 10⁶ to about 10¹⁰, from about 10⁶ to 10⁷, from about 10⁶ to 10⁸, from about 10⁷ to 10⁸, from about 10⁷ to 10¹, or from about 10⁸ to 10¹ cells). In certain embodiments, a single dosing unit of the composition comprises about 10⁶, about 107, about 10⁸, about 10⁹, or about 10¹⁰ or more cells. The patient may be administered with the pharmaceutical composition once every two days, once every three days, once every four days, once a week, once every two weeks, once every three weeks, once a month, or at another frequency as necessary to establish a sufficient population of engineered Treg cells in the patient.

Pharmaceutical compositions comprising any of the zinc finger nucleases or other nucleases and polynucleotides as described herein are also provided.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### Examples

### Example 1: Integrating Transgene into the AAVS1 Gene Locus of iPSCs

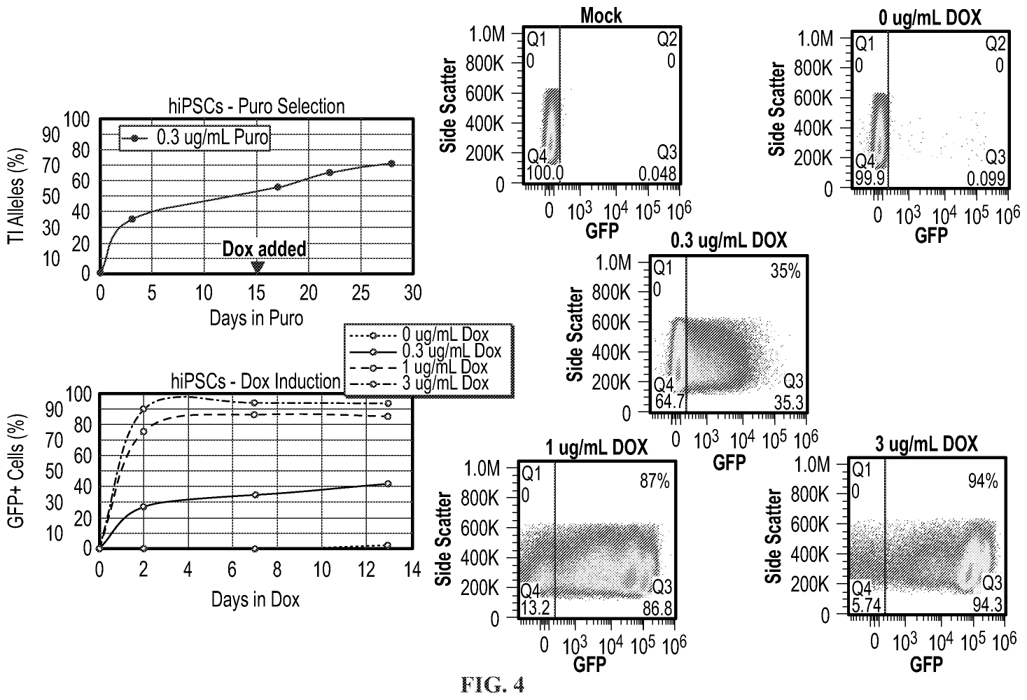

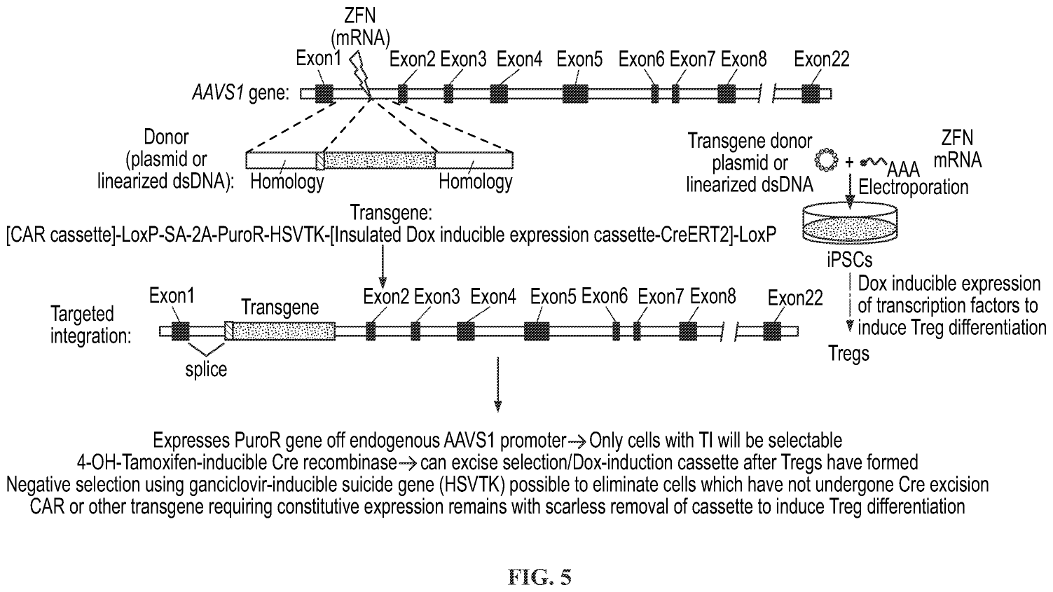

This Example describes an experiment in which a green fluorescent protein expression cassette was integrated into the AAVS1 gene locus as illustrated in FIG. 3. AAVS1 ZFN mRNA and donor plasmid were delivered into iPSCs via electroporation on Day −7. One week later (Day 0), puromycin was added (0.3 μg/mL) to the tissue culture to begin positive selection for cells having undergone targeted integration. Doxycycline was added at Day 15 and maintained in culture at 3 different doses (0.3, 1, and 3 μg/mL) to induce expression of the dox-inducible GFP expression cassette. Control cells did not have added doxycycline in culture. Cells were maintained in the presence of doxycycline for 13 days. During this period, the 3 μg/mL dose of doxycycline yielded the highest level of inducible GFP transgene expression (94%; FIG. 4). This high level of expression was sustained while doxycycline was present in culture. Cells were maintained in puromycin as well as doxycycline from Day 15-28 and were further positively selected (increase from ˜50% to ˜70% of alleles with targeted integration).

### Example 2: Skewing Differentiation of iPSCs Towards CD4⁺ Treg Lineage

To skew the differentiation of iPSCs toward becoming CD4⁺ T cells and ultimately Treg cells, the stem cells were subjected to blockage of signaling through IL-7 receptor by using an antibody targeting the alpha unit of the IL-7 receptor (IL-7Ra). Anti-IL-7Ra antibody was added to the cell culture media at increasing concentrations during the later stages of T cell development. Two duplicate experiments (Expt. 1 and Expt. 2) both show that addition of anti-IL-7Ra antibody increased the percentage of CD4⁺ single positive cells (bottom right quadrants), reaching 6.9% (Expt. 1) or 7.7% (Expt. #2), as compared to 2.81% or 4.78% for untreated cells, while reducing the percentage of CD8⁺ single positive cells (top left quadrants) (FIG. 9).

### Example 3: Generation of T Cells from TiPSCs and CD34-Derived iPSCs

This Example describes a study comparing the efficiency of obtaining differentiated T cells from iPSCs that were reprogrammed from mature T cells (Tregs, CD4⁺, and CD8⁺ cells) (termed “TiPSCs” herein) versus iPCSs that were reprogrammed from CD34⁺ HSPCs.

To reprogram T cells and CD34⁺ HSPCs, peripheral blood mononuclear cells (PBMCs) were obtained from healthy human donors through leukapheresis. T cell subsets were sorted on a flow cytometer (Sony SH800) following prior magnetic antibody-mediated enrichment for bulk T cells via CliniMACS® (Miltenyi Biotec) to obtain naïve CD4⁺CD25^{high}CD127^{low}CD45RA⁺ Tregs, bulk CD4⁺ T cells, and bulk CD8⁺ T cells. These T cell subpopulations were reprogrammed using a Sendai Virus based reprogramming kit (Thermo Fisher Scientific). CD34⁺ cells were enriched from the PBMCs through CliniMACS®.

Analysis was performed in at least two experiments in at least two different clones derived from naïve Tregs, CD4⁺ T cells, CD8⁺ T cells, or CD34⁺ stem cell. The iPSCs were allowed to differentiate to T cells over a period of 56 days.

The data in FIG. 12 demonstrate that the TiPSCs efficiently differentiated into cells expressing both CD3 and TCRαβ (Panels A and B). Co-expression of both T cell markers exceeded 20% in TiPSC lines. By contrast, only 5% of the cells differentiated from the CD34-derived iPSCs lines expressed CD3 and TCRαβ. P-values are as follows: naïve Treg=0.03, CD4=0.48, CD8=0.02.

Differentiated iPSCS generated various subpopulations of T cells when gating from live/single cells (FIG. 12, Panel C). The subpopulations of T cells were CD3⁺ TCR αβ⁺ cells and included CD4sp, CD8sp, double positive (CD4⁺CD8⁺), and double negative cells. The data show that only naïve Treg- and CD8-derived iPSCs generated significantly less double negative cells than CD34-derived iPSCs. P-values are as follows: naïve Treg=0.004, CD8=0.03.

By contrast, no subpopulations expressing CD3 and TCRαβ could be generated from CD34-derived iPSCs (FIG. 12, Panel D). The data show that CD4-derived iPSCs generated significantly more CD8sp cells that were also CD3⁺ TCRαβ⁺ than CD34-derived iPSCs (P-value=0.02).

### Example 4: FOXP3 and Anti-HLA-A2 CAR Expression in iPSC-Derived T Cells

This Example presents data on a gene editing study using an approach illustrated in FIG. 2. In the present study, the edited iPSC TRAC locus contained, from 5′ to 3′, (i) the TRAC exonic sequences 3′ to the integration site (i.e., the remaining exon 2 sequence downstream of the integration site, and the entire exon 3 sequence); (ii) a coding sequence for T2A; (iii) a coding sequence for (a) FOXP3/Helios/CAR, (b) FOXP3/CAR, (c) FOXP3, or (d) GFP; and (iv) a polyA site. The TCR alpha chain and the transgenes were all expressed under the control of the endogenous TCR alpha chain promoter. For clarity, all transgene coding sequences contained an in-frame 2A self-cleaving peptide coding sequence between neighboring transgenes to allow for polycistronic expression.

The edited iPSCs were differentiated to CD34⁺ hematopoietic stem progenitor cells (HSPCs) using an embryoid body method, followed by differentiation towards DP T cells using the StemSpan™ T Cell Generation kit (StemCell Technologies) (2 weeks of expansion and 1 week of maturation on LDCM). DP T cells were differentiated further by stimulation with a soluble CD3/CD28/CD2 activator. CAR expression was assayed by incubating cells with fluorescently tagged HLA-A2 dextramer.

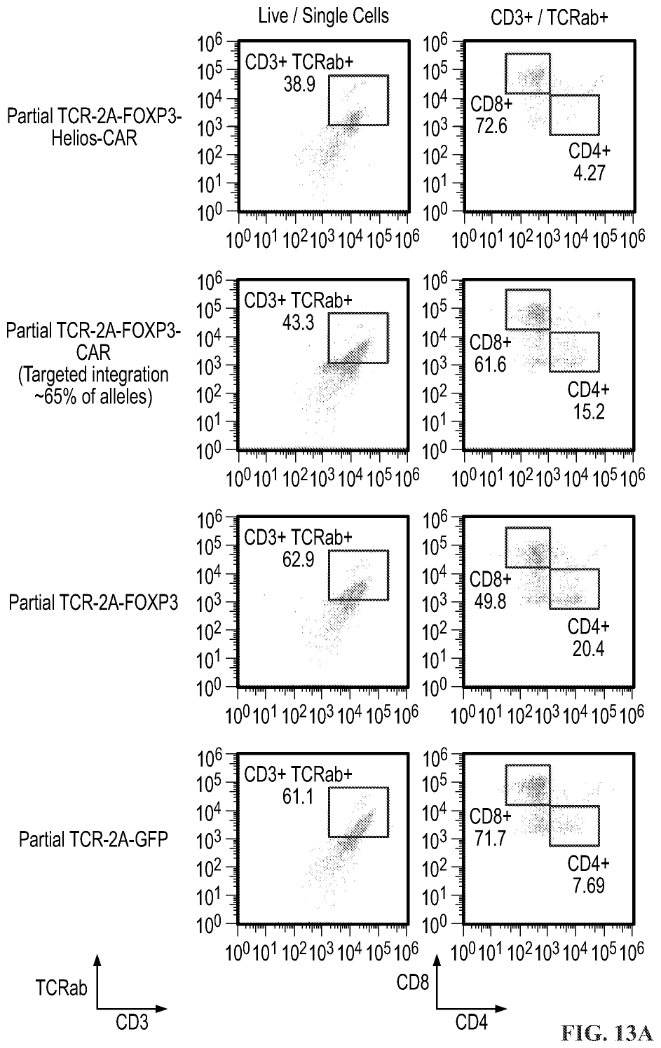

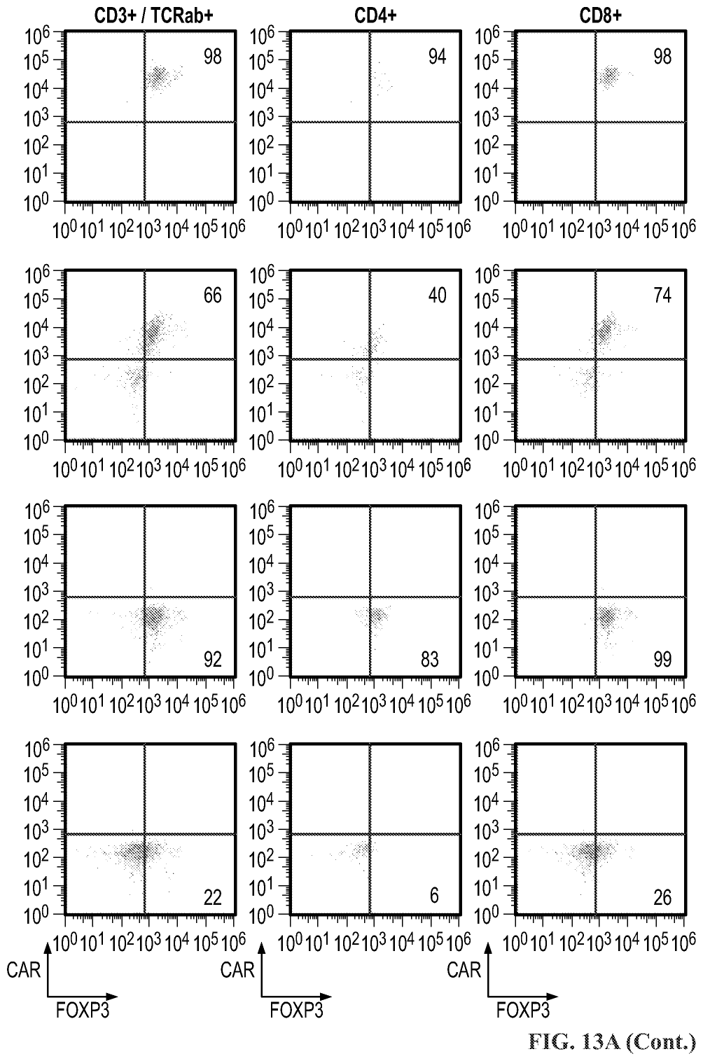

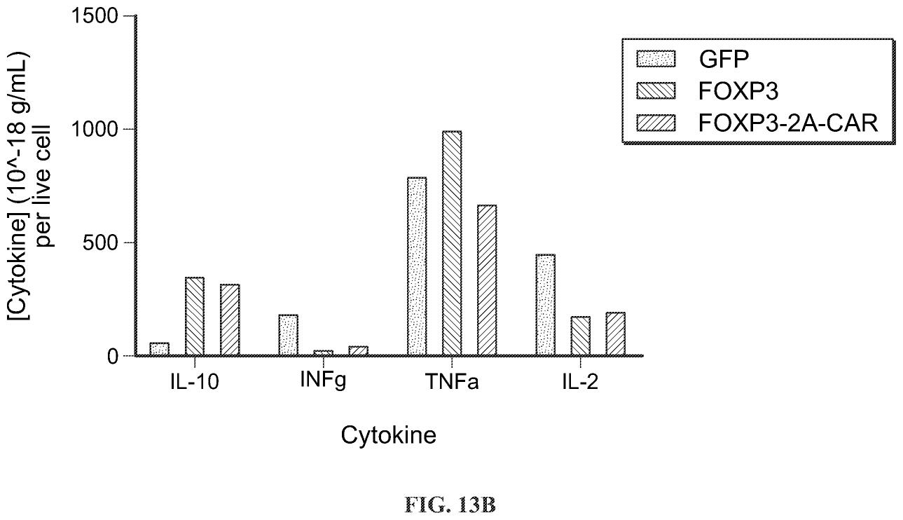

The data show that in the iPSC-derived T cells edited at the TRAC locus, the partial TCR coding sequence introduced by the transgene construct was able to maintain TCRαβ expression, and the FOXP3 and CAR transgenes were overexpressed in these cells as well (FIG. 13A).

The iPSC-derived T cells were further evaluated for their cytokine production profiles. The cells were cultured in 200 μL X-VIVO™ medium (Lonza) for 3 days prior to analysis of cytokine secretion (IL-10, IFN-γ, TNF-α, and IL-2) on a Luminex FLEXMAP 3D® instrument. Cytokine concentrations were normalized to total live cells seeded into culture. The data show that in iPSC-derived T cells containing the edited-in FOXP3 or FOXP3-2A-CAR transgene construct, there was an increase in the secretion of IL-10, an immunosuppressive cytokine associated with suppressive function of regulatory T cells through inhibition of differentiation/activation of effector T cells (FIG. 13B). Although no major differences in TNF-α secretion was observed, IL-2 and IFN-γ secretion was decreased in cells containing FOXP3 and FOXP3-2A-CAR transgene constructs. IL-2 is important in promoting the survival and proliferation of effector T cells, and the depletion of IL-2 is one mechanism through which regulatory T cells achieves its suppressive function. IFN-γ production by activated T cells has been shown to be suppressed by regulatory T cells. Thus, the results here demonstrate that FOXP3 overexpression from a FOXP3 transgene edited into the endogenous TRAC locus was able to confer a Treg-like phenotype to the edited T cells. The edited cells could express both the endogenous TCR as well as the CAR (where CAR was part of the transgene construct).

1. A genetically engineered mammalian cell comprising a heterologous sequence in the genome,
wherein the heterologous sequence comprises a transgene encoding a lineage commitment factor, and
wherein the lineage commitment factor promotes the differentiation of the cell to a CD4⁺ regulatory T cell (Treg) or promotes the maintenance of the cell as a CD4⁺ Treg.

2. The cell of claim 1, wherein the heterologous sequence is integrated into a T cell specific gene locus such that expression of the transgene is under the control of transcription-regulatory elements in the gene locus.

3. A method of making a genetically engineered mammalian cell, comprising:
contacting a mammalian cell with a nucleic acid construct comprising (i) a heterologous sequence and (ii) a first homologous region (HR) and a second HR flanking the heterologous sequence, wherein
the heterologous sequence comprises a transgene,
the first and second HRs are homologous to a first genomic region (GR) and a second GR, respectively, in a T cell specific gene locus or a genomic safe harbor locus in the mammalian cell; and
culturing the cell under conditions that allow integration of the heterologous sequence between the first and second GRs in the T cell specific gene locus or genomic safe harbor locus.

4. The method of claim 3, wherein the integration is facilitated by a zinc finger nuclease or nickase (ZFN), a transcription activator-like effector domain nuclease or nickase (TALEN), a meganuclease, an integrase, a recombinase, a transposase, or a CRISPR/Cas system.

5. The method of claim 3 or 4, wherein the nucleic acid construct is a lentiviral construct, an adenoviral construct, an adeno-associated viral construct, a plasmid, a DNA construct, or an RNA construct.

6. The cell or method of any one of the preceding claims, wherein the transgene comprises a coding sequence for an additional polypeptide, wherein the coding sequence for the lineage commitment factor and the coding sequence for the additional polypeptide are separated by an in-frame coding sequence for a self-cleaving peptide or by an internal ribosome entry site (IRES).

7. The cell or method of claim 6, wherein the additional polypeptide is another lineage commitment factor, a therapeutic protein, or a chimeric antigen receptor.

8. The cell or method of any one of the preceding claims, wherein the heterologous sequence is integrated into an exon in the T cell specific gene locus and comprises:
an internal ribosome entry site (IRES) immediately upstream of the transgene; or
a second coding sequence for a self-cleaving peptide immediately upstream of and in-frame with the transgene.

9. The cell or method of claim 8, wherein the heterologous sequence further comprises, immediately upstream of the IRES or the second coding sequence for a self-cleaving peptide, a nucleotide sequence comprising all the exonic sequences of the T cell specific gene locus that are downstream of the integration site, such that the T cell specific gene locus remains able to express an intact T cell specific gene product.

10. The cell or method of any one of the preceding claims, wherein the T cell specific gene locus is a T cell receptor alpha constant (TRAC) gene locus.

11. The cell or method of claim 10, wherein the heterologous sequence is integrated into exon 1, 2, or 3 of the TRAC gene locus.

12. The cell or method of any one of the preceding claims, wherein the transgene encodes FOXP3, Helios, or ThPOK.

13. The cell or method of claim 12, wherein the transgenes comprises a coding sequence for FOXP3 and a coding sequence of ThPOK, wherein these two coding sequences are in-frame and are separated by an in-frame coding sequence for a self-cleaving peptide.

14. The cell or method of any one of the preceding claims, wherein the cell is a human cell.

15. The cell or method of any one of claims 1-14, wherein the cell is a stem or progenitor cell, optionally selected from embryonic stem cell, induced pluripotent stem cell, mesodermal stem cell, mesenchymal stem cell, hematopoietic stem cell, a lymphoid progenitor cell, or a progenitor T cell.

16. The cell or method of claim 15, wherein the cell is reprogrammed from a T cell, optionally a Treg, a CD4⁺ T cell, or a CD8⁺ T cell.

17. The cell of any one of claims 1-14, wherein the cell is a Treg.

18. A method of producing the Treg of claim 17, the method comprising:
culturing the cell of claim 15 or 16 in a tissue culture medium that comprises (i) a low IL-2 dose, (ii) an inhibitor of IL-7Ra (CD27) signaling, (iii) an inhibitor of CCR7 signaling.

19. A method of producing the Treg of claim 17, the method comprising co-culturing the cell of claim 15 or 16 with MS5-DLL1/4 stromal cells; OP9 or OP9-DLL1 stromal cell; or EpCAM⁻CD56⁺ stromal cells.

20. The cell or method of any one of the preceding claims, wherein the cell comprises a null mutation in a gene selected from
a Class II major histocompatibility complex transactivator (CIITA) gene,
an HLA Class I or II gene,
a transporter associated with antigen processing,
a minor histocompatibility antigen gene, and
a β2 microglobulin (B2M) gene.

21. The cell or method of any one of the preceding claims, wherein the cell comprises a suicide gene optionally selected from an HSV-TK gene, a cytosine deaminase gene, a nitroreductase gene, a cytochrome P450 gene, or a caspase-9 gene.

22. A genetically engineered mammalian regulatory T cell (Treg) produced by the process of claim 18 or 19.

23. A method of treating a patient in need of immunosuppression, comprising administering to the patient a cell of any one of claims 1, 2, 6-17, and 20-22.

24. Use of the cell of any one of claims 1, 2, 6-17, and 20-22 in the manufacture of a medicament in treating a patient in need of immunosuppression.

25. A cell of any one of claims 1, 2, 6-17, and 20-22 for use in treating a patient in need of immunosuppression.

26. The method, use, or cell for use of any one of claims 23-25, wherein the patient has an autoimmune disease.

27. The method, use, or cell for use of any one of claims 23-25, wherein the patient has received or will receive tissue transplantation.

28. The method, use, or cell for use of any one of claims 23-27, wherein the patient is a human.
